(19) United States
(12) Patent Application Publication
(10) Pub. No.: US 2014/0243395 A1
(43) Pub. Date: Aug. 28, 2014

(54) SPRAY SYSTEM FOR PRODUCTION OF A MATRIX FORMED IN SITU

(71) Applicant: ETHRIS GMBH, Martinsried (DE)

(72) Inventors: Carsten Rudolph, Munchen (DE); Senta Üzgün, Mainz (DE)

(73) Assignee: ETHRIS GMBH, Martinsried (DE)

(21) Appl. No.: 14/347,920

(22) PCT Filed: Sep. 25, 2012
(86) PCT No.: PCT/EP2012/068889

§ 371 Date: Mar. 27, 2014

(30) Foreign Application Priority Data
Sep. 28, 2011 (DE) 10 2011 114 986.8

(51) Int. Cl. A61K 47/34; A61K 31/713

(52) U.S. Cl. CPC A61K 47/34; A61K 31/713
USPC 514/44 A; 514/772.3

(57) ABSTRACT

A spray system for production of a matrix formed in situ is described, which comprises at least one lipophilic component comprising at least one polymer based on glycolic acid and lactic acid and at least one biocompatible solvent having an X log P3 value of less than 0.2, and at least one hydrophilic component, wherein the at least two components are present separately from each other prior to use and are not mixed until or in the course of spraying, with components forming a film when sprayed onto human tissue.

The invention relates to a spray or application system to be used for preventing adhesions, in particular surgical adhesions.

After injuries and surgery, adhesions frequently form. They develop into accretions and scars and lead to post-operative complications. In particular, surgical interventions in the abdomen lead to primary post-operative adhesions in up to 94% of the patients. The peritoneum forms as a serous membrane the lining of the abdominal cavity. It consists of a visceral and a parietal layer with a serous gap which is filled with from 5 to 20 ml liquid and allows a free sliding movement of the organs. Histologically, the peritoneum comprises a single layer of squamous epithelium, also called mesothelial layer, and a thin layer of subserous connective tissue. Within a few days, injury of the mesothelial layer results in the formation of permanent adhesions between the two layers and the surrounding tissue. Besides injury resulting from surgery, the mesothelial layer may already be injured by the use of swabs, the drying out of the surface during surgery or by contact with talcum via talcum-powdered gloves. Even in minimal-invasive surgery, such as laparoscopy, the activation process is set in motion.

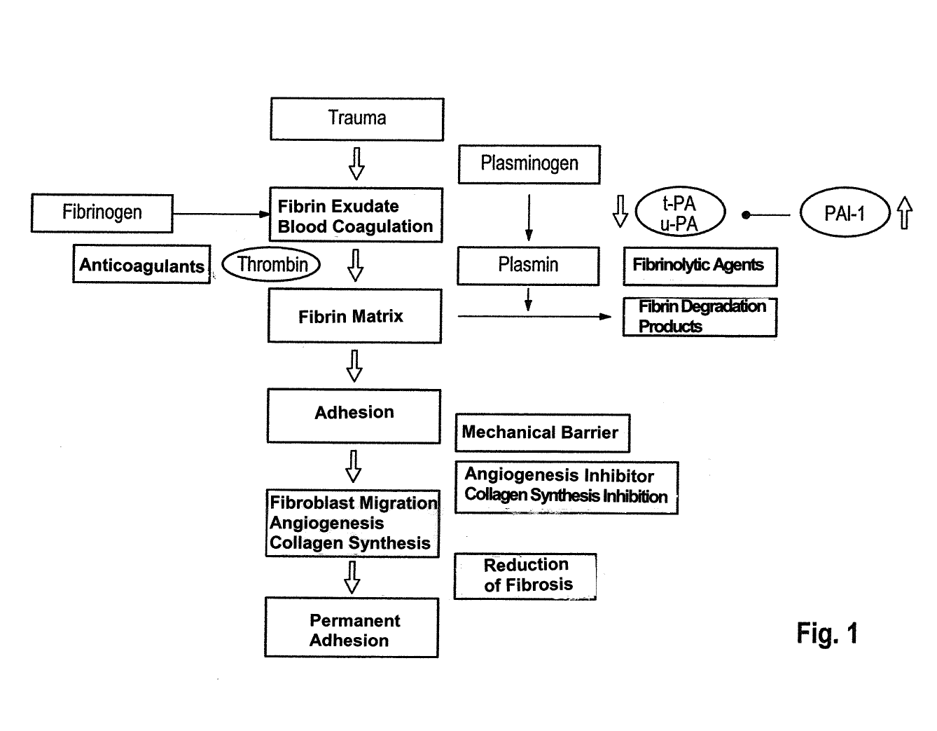

Inspite of more than a century of research in the pathophysiology of peritoneal adhesions, to this day the findings are still incomplete. FIG. 1 shows in summary the pathogenesis of peritoneal adhesions with possible therapeutic approaches. It is assumed that traumatization of the peritoneal tissue causes an inflammatory reaction with exsudation of inflammatory cells and soluble fibrin monomers. These form fibrous structures within about 3 hours, which may be dissolved within the first few days by the serine protease plasmin if there is sufficient fibrinolytic activity. However, if this does not happen, as a consequence collagen-rich connective tissue, i.e. permanent adhesion, will form, which will then cause problems.

While in the first two days after injury, mainly neutrophilic leukocytes are involved in the inflammatory process, macrophages and mesothelial cells play an important role in the genesis of permanent adhesion. Both cell types are capable of releasing plasminogen, a precursor of plasmin, into the blood stream. In the capillaries, plasminogen is transformed into plasmin by the serine protease plasminogen activator (tissue/urokinase plasminogen activator, t-PA/u-PA). These proteases are likewise secreted by the mesothelial layer. Triggered by an increased concentration of inflammatory cytokines, such as the tumor necrosis factor (TNF), the transforming growth factor (TGFβ), or interleukins, the active tPA concentration decreases in the posttraumatic phase. This leads to a significant reduction of the fibrinolytic activity in the abdominal cavity, which results in an imbalance between fibrinolysis and fibrin formation and promotes the formation of permanent adhesions. The decrease in the active t-PA concentration in tissue is, in turn, the consequence of a reduced t-PA production in the mesothelial cells and a simultaneous hyperexpression of plasminogen activator inhibitor type 1 (PAI-1), the most important inhibitor of the tissue plasminogen activator. Similar to primary wound closure, where thrombozytes increasingly secrete PAI-1 to thereby prevent a premature lysis of the fibrin and thus to initiate primary wound closure, in the posttraumatic phase it comes to an increased formation of the plasminogen activator inhibitor by mesothelial cells and endothelial cells of submesothelial blood vessels. It is therefore assumed that the t-PA/PAI-1 balance is the key point for the formation of peritoneal adhesions.

For the therapy of permanent adhesion there are different approaches, such as:

i) primary prophylaxis by avoiding injuries and inflammations,
ii) prevention of coagulation of serum-containing exudate by anti-coagulants,
iii) dissolution of the fibrinous structures by fibrinolytic agents,
iv) use of mechanical barriers until regeneration of the mesothelial layer by separation, and
v) prevention of fibrosation.

The use of fibrinolytic agents and the use of physical barriers were considered promising therapeutical approaches; however, none of these approaches has found clinical acceptance in view of the disadvantages associated with them. It has been found that the presently available fibronolytic agents have an insufficient anti-adhesive activity, presumably due, inter alia, to their short half-life in the plasma. The consequently required high dosages produce strong side effects preventing their use. Known fibrinolytic agents are streptokinase, urokinase and the recombinantly produced t-PA protein alteplase (obtainable as Actilyse®), and its modified form reteplase (commercially available form Rapilysin®). Alteplase has a half-life in plasma of 3 to 6 minutes only, which for the modified form reteplase could be increased to 13 to 16 minutes. Therefore, multiple applications and infusion pumps are required to obtain continuous drug levels, which produce high side effects.

Physical barriers, too, have already been used for reducing adhesions. However, so far it has not been possible to show a positive effect on postoperative complications. The presently available physical barrier systems are limited to the local field of application. Known physical barriers are mainly absorbable tissues, such as oxidized cellulose fibres, a combination of hyaluronic acid and carboxymethyl cellulose, or PEG gels.

For example, it is known from US 2011/0052712 A1 to use biodegradable polymers as adhesion barriers. This document suggests a formulation for generating an adhesion barrier that includes a large number of particles from a polymer combination of a biodegradable polymer and at least one water soluble polymer, which is deposited on a tissue in form of a film so as to prevent adhesion. The water soluble polymer after application is intended to absorb water from the tissue, to swell, thus allowing film formation and the provision of water so that the particles gradually decompose and release the possibly included active agent. These particles may contain as active agent, e.g., an anti-inflammatory agent. However, the properties of the films obtained with this formulation depend on the amount of water available at the site of application and cannot be adjusted in a reproducible manner.

It has also been suggested to apply to the site of injury or surgery active agents which should prevent adhesion. Since liquids do not remain long at the desired place, in the past systems have been developed where active agents, possibly encapsulated in biodegradable polymers, are provided in a prefabricated matrix. However, fixed systems are uncomfortable for patients, especially in the area of the abdominal cavity. Therefore, it has also been suggested that instead gels be used, that may contain active agents. Thus, WO 2004/011054, for example, discloses a polymer depot composition comprising a polymer matrix from different types of polymers with low to high molecular weights, which includes a solvent hardly miscible in water to improve the plasticity of the polymer. The suggested composition is a complex system of various types of polymers and therefore expensive and complex in production and use.

A disadvantage of the known systems using water from the surroundings for matrix formation by containing a water soluble or water swellable polymer for absorbing water into the matrix consists in that an active agent included in the matrix is too rapidly released by the water so that initially there is too high a concentration of active agent at the site of action. Desirable is a uniform release without a so-called “burst” at the beginning. To achieve this object it was suggested in US 2009/0004273 to encapsulate proteins and peptides by using a polymer system which does not form a hydrogel when the system comes in contact with tissue fluid. To bring about a continuous linear release of the active agent and to prevent a burst at the beginning, two different polymer systems consisting of a hydrophobic component and a hydrophilic component are used, which may, for example, be supplied in the form of a film or a coating of devices.

To flexibilize the applicability of implants it was also suggested to form films or implants in situ. In this connection, DE 100 01 863, for example, describes implants that are formed in situ by mixing a carrier material and a solvent shortly before application, so that at least some of the carrier material is dissolved so as to then form liquid crystalline phases in the body. The carrier material is provided in powdered form and obtained, e.g. by spray drying. In particular when it additionally includes an active agent care must be taken that the carrier material is sufficiently mixed for distributing the active agent uniformly in the produced matrix.

Further carrier systems formed in situ have been described for the production of implants. Since it is not possible to use changes in temperature and pH values as well as reactive components for matrix formation directly in the body, the most frequently used technology is solvent precipitation. Therefore, in the known processes the implant is most often solidified through the contact of a water-insoluble polymer, dissolved in an organic solvent, with the tissue fluid (lymph). To accelerate precipitation, in some of the known processes, as discussed above, water-absorbing components are added to the composition, such as swellable polymers. Depending on the carrier system and the organic solvent used, it either comes to an increase in viscosity with formation of a viscous gel or to a genuine precipitation of the carrier system with matrix formation. For this, a copolymer of lactic acid and glycolic acid is frequently used, the precipitation of which may be controlled by solvent and polymer selection. Depending on the molecular size of the pharmaceutically active component, both release kinetics and release duration may be adjusted as appropriate. Two technologies described in the prior art are the Atrigel® technology which uses N-methyl-2-pyrrolidone as water-miscible solvent, and the Alcamer® technology employing hardly water-miscible solvents. It is a well-known fact that a higher water miscibility leads to a faster implant formation and thus to a higher porosity of the matrix, while hardly water-miscible solvents or highly concentrated polymer solutions lead to slower implant formation. The former approach leads to rapid release of the embedded components and also to a higher initial release of the active agent, the so-called “burst”. The latter approach leads to sustained release, with release only starting after some time. A product based on the Atrigel® technology is commercially available in the form of a hormone preparation for the treatment of advanced hormone-related prostate cancer.

Such systems are advantageous in that they can be applied directly at the desired site and that an active agent may also be embedded into the matrix during application. The largest problem with the known implants formed in situ is, however, morphology control of the implant and thus control of drug release. The morphology of the implant is dependent on the conditions at the site of application, whereby reproducibility becomes almost impossible and predetermined setting of the release kinetics is prevented.

Thus, all previously known systems still have disadvantages and are not yet satisfactory in use. Therefore, it was an object of the present invention to provide a system that overcomes these disadvantages, is easy to use, does not require complicated or expensive measures during use, and helps to reliably prevent adhesions after injuries and surgery. Furthermore, the system should provide the possibility of delivering an active agent, wherein the release of the active agent should be predictable, adjustable and occur in a constant manner, without causing a burst at the beginning, but also without an unduly long delay.

Furthermore, it was an object of the present invention to provide an application system that can be directly sprayed onto the envisaged site, that is capable of absorbing active agents, in particular hydrophilic agents, such as nucleic acids, proteins or peptides, and of releasing them in a controlled manner, that can produce a stable film at the site of application, with the release properties thereof being adjustable and optimizable. Moreover, an application system should be provided that is physiologically compatible and does not hinder the activities of proteins, peptides and nucleid acids, thus allowing the release of active products.

In addition, it was an object of the present invention to provide an application system that can effectively prevent surgical adhesions and help prevent permanent adhesions.

The above-mentioned objects are achieved with a sprayable application system as defined in claim 1. The sprayable application system, hereinafter also referred to as spray system, comprises at least one lipohilic component which is formed from at least one polymer dissolved in a solvent, and one aqueous component, as well as optionally at least one active agent. It may comprise further components.

It was surprisingly found that the specific composition as defined in the present invention provides a carrier material that is easy to use, is stable, can be applied onto the desired site and to the desired exent, and that is capable of providing an active agent for the desired period of time and at the desired rate of release.

The advantageous properties are achieved with a spray system comprising two components, with the one component having at least one polymer dissolved in a solvent, and the other component having at least one aqueous solvent, with the components being blended with each other directly before or during application and being applied by spraying, with the components of the invention forming a matrix in situ which decomposes after a predetermined period of time, and, in this period of time, releases the optionally included active agent in a controlled manner.

The film formed with the system according to the present invention has a high quality and, for a pretermined time, remains at the site of application, where it exerts its effect. Only with the combination of the features of the invention is it possible to obtain a carrier material having the desired properties.

Important features of the present invention are polymer type and solvent type as well as the form of application, i.e. bringing the two components into contact directly before or at spraying or during spraying.

One of the essential features of the system according to the present invention is the lipophilic component which comprises at least one polymer based on glycolic acid and lactic acid, and at least one biocompatible solvent for said polymer, the solvent having a predetermined log P value, as explained in more detail below. This lipophilic component is then blended with at least one hydrophilic component comprising at least one aqueous solvent directly before or at spraying, which by precipitation of the polymer produces a matrix that forms a physical barrier at the site of spraying and which is capable of effectively preventing surgical adhesions. In a preferred embodiment, the lipholic component and/or the hydrophilic component comprise(s) at least one active agent which, during spraying and film formation, is embedded into the film and released therefrom in a controlled manner, and which additionally blocks surgical adhesions by physiological and/or biological means.

The particular advantage of the present application system consists in that, due to the selection of the specific applied components, a polymer matrix is formed during spraying by precipitation of the polymer from the solvent. In this polymer matrix an active agent is embedded if the composition contains one. Precipitation of the polymer shall mean that the solubility limit of the polymer is exceeded and that the polymer is no longer dissolved or completely dissolved in the solvent. By adjusting the individual components it is possible to predetermine the precipitation rate and the film formation rate and thus the porosity of the resulting matrix, which results in controlled release characteristics. In view of the variability of these polymers, there are many possibilities to adjust the optimal properties; however, it is not always easy to find the optimum solution for the specific case. Therefore, parameters allowing the selection of an optimum system are described in the following.

Critical factors for the system of the invention are mainly: the polymer used, the solvent used for dissolving the polymer, the contents of polymer, solvent and aqueous component, as well as, optionally, the content and form of the active agent.

The material used for matrix formation should be sterilizable, and it must allow a controlled release of a contained active agent over a period of time in which adhesion formation or scar formation may occur. This is a period of time in the range of from at least two weeks up to six weeks, and preferably of from two to four weeks. Furthermore, the material must have a quality such that it retains it stability for a time sufficient for achieving the desired object, i.e. for preventing adhesions.

An important parameter for the selection of the suitable polymer is molar mass (molecular weight). The selection of the suitable molecular size is made on the basis of the inherent viscosity (natural logarithm of the relative viscosity based on the concentration C of the dissolved substance). In a manner known per se, the inherent viscosity of the PLGA polymers is measured with 0.1% in CHCl₃ at 25° C. For the system of the invention such polymers are preferred that have an inherent viscosity in the range of from 0.1 to 0.8, in particular of from 0.15 to 0.7. If the value is below 0.1, the polymers are frequently too small to sustain a sufficiently long activity. If the value is too large, a sufficient quality of the film cannot be guaranteed; moreover, the delay until release starts may be too long. To achieve optimum properties it is also possible to use mixtures of polymers with different molar masses.

The molar mass of the PLGA polymers may also be determined by conventional methods, e.g. by gel permeation chromatography. It was found that PLGA polymers having a molar mass in the range of from 10 to 63 kDA are well suited.

There are further factors that are helpful for selecting a polymer suitable for the application system of the present invention. The system of the present invention must be sprayable which implies that it must be soluble or suspendable in a biocompatible solvent.

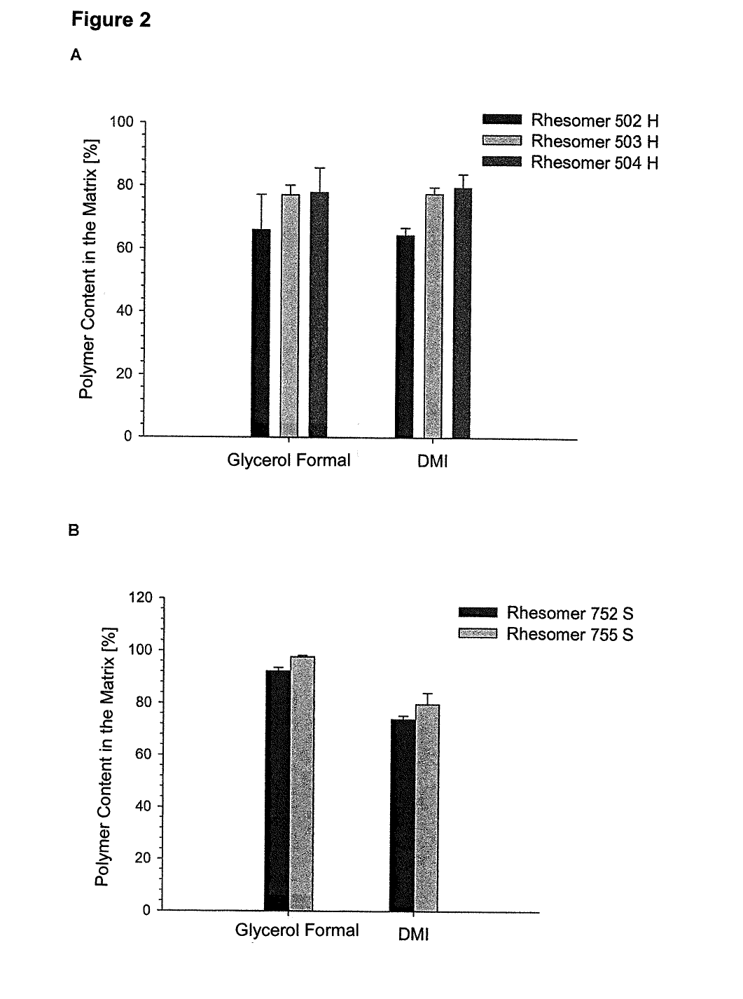

A measure of the quality and mechanical stability of the film formed from the inventive application system is the quality of the matrix and/or the film, which can be determined with the methods described in the Examples. FIG. 2 shows the results of the tests described in the Examples with respect to the film quality of a number of combinations of polymers and solvents. The film quality is essentially determined by the choice of solvent and the molar mass of the polymer. For its determination, the percentage of the polymer in the supernatant (loss) and in the precipitate (matrix quality) based on the total amount of polymer used is ascertained. The matrix quality of the resulting layer or the resulting film is critical for the system of the invention, since the system must function for at least two and up to six weeks. This is only possible if the formed film or the formed layer is sufficiently stable and at the same time provides the desired release kinetics. Thus, the matrix quality should lie in a range of from 80 to 100%, preferably of from 90 to 100%, and most preferably of from 95 to 100%, with the value being determined at room temperature, i.e. at approximately 25° C., with the methods described in the Example.

The matrix quality depends inter alia on the molar mass of the polymers. It has been found that with polymers having a higher molar mass it was possible to incorporate a larger amount of polymer into the matrix, whereas with polymers having a lower molar mass there was a loss of polymer (for film formation). For example, it was found that for a PLGA polymer having a ratio of lactide to glycolide of 75:25 and an inherent viscosity of from 0.5 to 0.7, i.e. having a comparatively high molar mass and using a polymer with esterified end groups, almost 100% of the amount of polymer used formed the matrix. In contrast, it was found for a PLGA polymer having a ratio from lactide to glycolide of 50:50 and an inherent viscosity of <0.6 and free end groups that polymer was lost during matrix formation, i.e. it was not embedded into the matrix. This effect may be heightened or improved by the solvent.

As stated above, the polymer used is one of the essential components of the system. In accordance with the invention, glycolic acid-based and lactic acid-based polymers, namely poly(lactide-co-glycolides), usually poly(D,L-lactide-co-glycolides), hereinafter also referred to as PLGA polymers, are used in the application system. It is possible to use polymers based on D,L-lactide and polymers based on the enantiopure L-lactide.

Lactic acid-based and/or glycolic acid-based polymers have been known for quite some time, also for systems of controlled release. Generally, PLGA polymers are processed to microparticles or implants which may then be used in various ways. PLGA polymers are biocompatible and biodegradable and their properties may be adapted to the respective purpose.

The present invention utilizes glycolic acid-based and lactic acid-based polymers that are dissolved in a solvent. The release kinetics of these polymers are adjusted by means of their molecular weight, molecular weight distribution, and their end groups.

Thus, by specifically selecting the polymer and the solvent it can be chosen whether the resulting matrix shall effect a diffusion-controlled, erosion-controlled, or both a diffusion-controlled and erosion-controlled release. A rapid matrix formation at high quality, as achieved in accordance with the invention, constitutes an important tool for achieving linear release kinetics without initial loss of active agent.

It has been discovered that PLGA polymers are more suitable for the inventive system than pure polylactide (PLA) or pure polyglycolide (PGA). By adjusting the ratio of lactic acid units to glycolic acid units, it is possible to precisely control the properties, in particular the degradation properties, in a manner known per se. In the application system of the invention such PLGA polymers have proven to be preferable that have a ratio of lactide units to glycolide units in the range of from about 75 to about 25 to from about 25 to about 75. The expression “ratio of lactide units to glycolide units” consistently refers to the molar ratio of the units in a polymer. It is also possible to use mixtures of various types of PLGA polymers. It is possible to use mixtures of any type of PLGA polymers, e.g. mixtures of polymers wherein the molar ratio of lactide units to glycolide units and/or the molar mass or the inherent viscosity and/or the kind of lactide units (D/L or L) and/or the end groups vary/varies. The mixture best suited for the particular purpose can be found with routine tests.

The degradation rates of the PLGA polymers are dependent on the content of PGA or PLA, with PLGA copolymers generally having shorter degradation rates than PLA polymers or PGA polymers. For this reason, PLGA polymers are preferred. The shortest degradation times are achieved with polymers having a ratio of lactide to glycolide of 50:50. Due to the additional methyl group in the lactic acid monomer, an increase of the PLA content impedes the hydrolysis of the polymer and, at the same time, increases the hydrophobicity, which leads to longer degradation times. Also an increase of the PGA content in the polymer or the use of the pure stereoenantiomere L-lactic acid compared to the monomer D-/L-lactic acid lengthens the degradation times of the polymer by increasing the crystallinity of the polymer, since water diffuses more easily into amorphous areas. Consequently, these areas are more rapidly degraded than crystalline ones. Thus, the crystallinity of the polymer steadily increases during degradation. By adjusting the ratio it is thus possible to set crystallinity and degradation time to predefined values. Furthermore, the degradation rates can be accelerated by shorter polymer chains and free end groups. Free end groups, i.e. free hydroxy groups and free carboxy groups increase the hydrophilic properties of the polymer, so that the diffusion rate of the water and its content in the polymer matrix increases. Furthermore, free carboxylic groups catalyze the hydrolysis of the polymers by lowering the pH value within the matrix. Thus, polymers having free end groups are preferably used in accordance with the invention.

In accordance with the invention it is preferred to use PLGA polymers having free end groups, which have a ratio of lactide units to glycolide units of from 40:60 to 60:40, more preferably of about 50:50 and/or which have an inherent viscosity of smaller than 0.6. When PLGA polymers having esterified end groups are used, such with a ratio of lactide units to glycolide units of 75:25 are preferred.

Suitable PLGA polymers are commercially available, such as resomer polymers (available from Evonik Industries AG, Essen, Germany), in particular resomers from the Resomer® H series or the Resomer® S series. Particularly well suitable polymers are, for example, Resomer® 502H, 503H and 504H or Resomer® RG755S. The following Table 1 lists some properties for preferred polymers:

TABLE 1
Properties of the used Resomer ® RG Polymers

| | | | Acid | | |
|---|---|---|---|---|---|
| | | | groups** | | |
| | | i.v.* | [mg | MwGPC*** | Release**** |
| Resomere ®RG | PLA/PGA | [42] | KOH/g] | [43] | [days] |
| 502H | 50/50 | 0.22 | 9 | n.s. | 20 |
| 503H | | 0.32 | 5 | 34 | 15 |
| 504H | | 0.51 | 3 | 48 | 70 |
| 752S | 75/25 | 0.21 | 0 | 12 | n.s. |
| 755S | | 0.63 | 0 | 63 | 30 |

*inherent viscosity (i.v.; 0.1% solution in chloroform at 25° C.)
**number of acid groups (potentiometric titration) taken from the manufacturer's information.
***molar mass, measured by GPC, and
****the release data originate from the publication of Eliaz et al. [44[Eliaz, 2000 #257]. The release data for Resomer ® RG 755 S and 503 H relate to the release of albumin from bovine serum [44] and for Resomer ® RG 502 H and 504 H to the release of thymus DNA [45] from an injectable implant (10% to 20% PLGA (m/v) in tetraglycol).

The polymer matrix is degraded via ester hydrolysis to the biocompatible monomers lactic acid and glycolic acid, which are subsequently metabolized, via the Krebs cycle, to CO₂ and water. The degradation pattern of the PLGA implants is based on bulk erosion which is characterized in that water diffuses faster into the polymer matrix than the polymer is degraded. Accordingly, this leads to a homogenous mass loss over the total cross section of the polymer matrix. The degradation process may generally be subdivided into three sections:

1. Hydration: The polymer absorbs water and swells, with a small fraction of ester bonds already being broken. However, a mass loss does not yet occur.
2. Degradation: The mean/average molar mass considerably decreases. The carboxylic acid groups produced upon cleavage of the ester bonds lead to a drop in the pH value within the matrix and consequently to autocatalysis of ester cleavage. The polymer loses in mechanical strength and/or mechanical stability.
3. Solution: Towards the end of degradation, the low-molecular fragments and oligomers dissolve in the surrounding medium, with the dissolved polymer fragments in turn being hydrolyzed to free carboxylic acids.

The degradation times are decisive for the release of encapsulated macro molecules and nano-scale carrier materials, since, in view of their size, they are predominantly released by matrix erosion so that it becomes possible to specifically control the release rates via the degradation rates. The degradation times of the PLGA polymers can be controlled by means of their composition and the molar mass of the polymers. In the case of the commercially available PLGA polymers, the inherent viscosity is generally indicated as dimension for the molar mass.

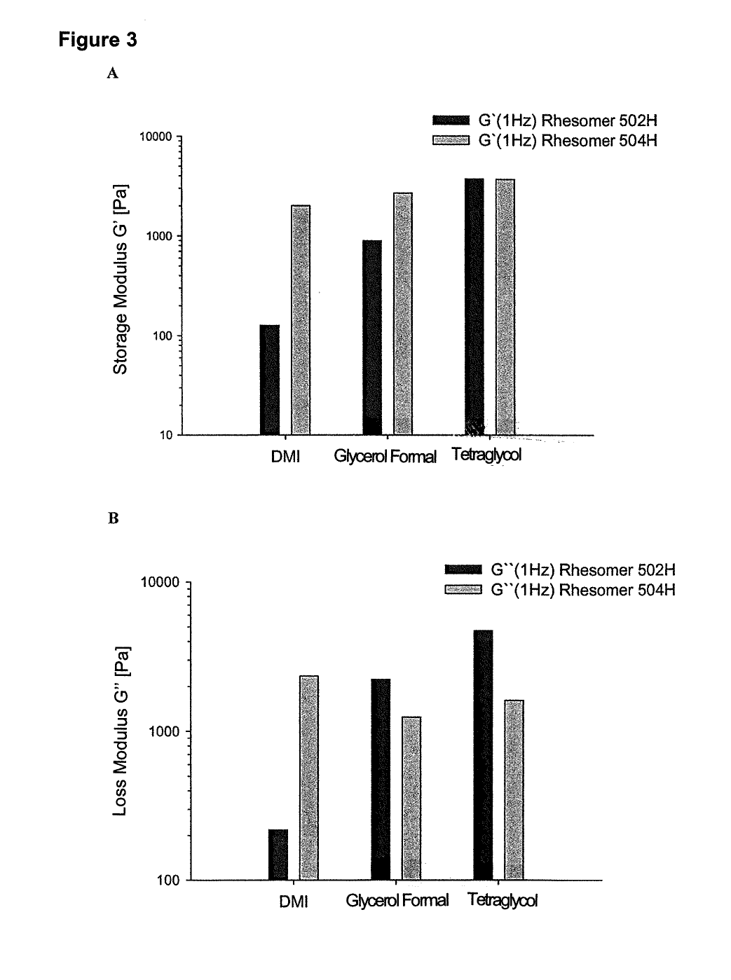

The viscoelastic properties of the system likewise play a role, as shown in FIG. 3 and in the Examples.

For the application system of the invention it is essential that a high quality material is produced which retains its mechanical strength and/or stability long enough for preventing adhesions and which is subsequently degraded. When the carrier system formed from the application system of the invention is loaded with active agent it is additionally necessary that the active agent is released with the desired release kinetics.

The matrix quality of the film obtained with the application system of the invention depends on the polymer used, the solvent used for its solution, and the water solubility thereof. It has been found that the solvent used for dissolving the PLGA polymer has a considerable effect on the quality of the matrix produced therewith. The matrix produced upon combination of the PLGA, dissolved in the solvent, with the aqueous phase thus is dependent on the type and amount of the solvent, in particular on its hydrophilic property.

On the other hand, the selection of the solvent is also dependent on the type of the polymer used. The more lipophilic the polymer, the more lipophilic the solvent must be. The lipophilic property of the polymer is, inter alia, dependent on its end groups, because a PLGA with free acid groups is more hydrophilic than a PLGA with esterified end groups.

On the one hand, the solvent must dissolve the selected polymer to such an extent that the polymer is sprayable, on the other hand, the solubility of the solvent in water must be high enough for precipitation to occur rapidly after spraying on of the two components. A useful parameter for selection of the suitable solvent is the log P value.

Since, as shown above, the matrix quality likewise changes depending on the molar mass of the polymer by the solvent, a further essential feature of the invention is the solvent. An important parameter for selecting the solvent is miscibility with water. The higher the miscibility with water, the faster the matrix formation, however, the porosity also increases. The lower the water miscibility, the slower the matrix formation, and the higher the quality.

The water miscibility of a solvent can be determined via the log P value.

The log P value indicates the octanol/water partition coefficient, i.e. the ratio of the concentration of the solvent in a two-phase system of 1-octanol and water. The log P value is defined as follows:

$$log P = log \frac{{c}_{0}^{{S}_{i}}}{{c}_{Ω}^{{S}_{i}}} log {c}_{0}^{{S}_{i}} - log {c}_{Ω}^{{S}_{i}} .$$

The calculation or determination of the log P value is known per se. An algorithm suitable for determining the log P value is X log P3, as described in Cheng et al. (Cheng T., Zhaoy, Lix, Lin F., Xu Y., Zhang X. et al., Computation of Octanol-Water Partition Coefficients by Guiding an Additive Model with Knowledge. J. Chem. Inf. Model. 2007; 47:2140-2148). The log P value calculated in this manner yields positive values for lipophilic substances and negative values for hydrophilic substances. It has been found that in the system of the present invention substances may be used being not very lipophilic, so that solvents are preferred having a negative or at least a very small positive X log P3 value.

Solvents with an X log P3 value of lower than 0.2, preferably of lower than 0, and in particular in the range of from −0.2 to −1.5, especially preferably solvents having an X log P3 value of between −0.25 and −1.0, have proven to be suitable for the system of the present invention. The X log P3 value should be the higher, the more lipophilic the polymer used.

It has been found that when a polymer solution wherein the solvent has a log P of lower than 0.2 is mixed with an aqueous component and sprayed onto the site of application, precipitation occurs in a predeterminable and reproducible manner, which produces a polymer film having the desired properties.

The more lipophilic the polymer used, the more lipophilic the solvent used must be, and the lower its water miscibility. The larger the difference between the solubility of the polymers in the solvent or water, the stronger the effect on the kinetics of film formation. Therefore, if the PLGA polymer used is one having esterified end groups and thus a higher lipophilic property, then the solvent used should likewise be more lipophilic. A more lipophilic solvent has poorer water miscibility and thus results in high matrix quality. It has been found that the best results are obtained when the system of polymer and solvent is close to the solubility limit and the solvent has the best possible water miscibility, so that upon addition of the aqueous phase film formation is rapid and complete, with a high percentage of the polymer being present in the matrix.

Solubility of the polymer in the solvent likewise plays a role. The better the polymer is dissolved in the solvent, the more water will later be required to precipitate the polymer from the film and to form a film. On the other hand, the solubility must be such that a sufficient amount of polymer can be dissolved in the solvent. It has been found that a solvent is suitable for forming a high quality film, which, for the PLGA to be used, has a solubility of at least 5% (mass/volume) (m/v), preferably of from 5 to 60%, and in particular a solubility of from 10 to 30%, at room temperature. The selection of a suitable solvent should be based on the following correlations: The solubility of a solvent for a polymer decreases with increasing molar mass of the polymer. The more lipophilic the polymer, i.e. the more esterified the polymer and/or the longer the polymer chain, the more lipophilic the solvent must be. Thus, when using a highly lipophilic polymer with a highly water-soluble solvent, the polymer will only be dissolved to a very small extent, while a less lipophilic polymer, e.g. a polymer having free acid groups and a lower molar mass, is readily soluble in a hydrophilic solvent. On the other hand, the better dissolved the polymer, the more water is required for precipitation. Very good results can be obtained when polymer and solvent are selected such that the solubility is from 5 to 15%, with the solvent having an X log P3 value in the range of from −0.3 to −1.0. In this combination, the polymer very quickly precipitates and forms a high-quality film when water is added. Solvents having an X log P3 value in this range are known to the person skilled in the art.

Therefore, a highly suitable solvent will combine good water miscibility with a polymer solubility such that, with the desired amount of polymer, the solubility in the solvent is close to the saturation limit at application temperature, i.e. from 30 to 40° C. In any case, the solubility at room temperature must be sufficiently high for forming a stable solution.

Tetraglycol, glycerol formal and dimethyl isosorbite (DMI) have been found to be particularly well suitable. The solvent tetrahydrofurfuryl alcohol polyethyleneglycol, also called tetraglycol or glycofurol, is a solvent that has long been in use for parenterals. Concentrations of up to 50% are used and in this dilution the solvent only shows low toxicity.

Glycerol formal is an odorless solvent with low toxicity consisting of a mixture of 1,3-dioxane-5-ol and 1,3-dioxolane-4-methanol. It is an excellent solvent for numerous pharmaceuticals and cosmetics. Especially in veterinary medicine it is used as solvent for injections. Glycerol formal is commercially available, e.g., as Ivumec® and PTH®. Ivumec™ at 0.27% has been approved for subcutaneous application in pigs and is normally used at 0.1 mg/kg.

Dimethyl isosorbide (DMI) is known for topical application. Commercially available preparations containing DMI are Mykosert® and Ibuprop-Gel®. DMI is topically used as penetration-enhancing substance. A low hemolytic activity has been observed.

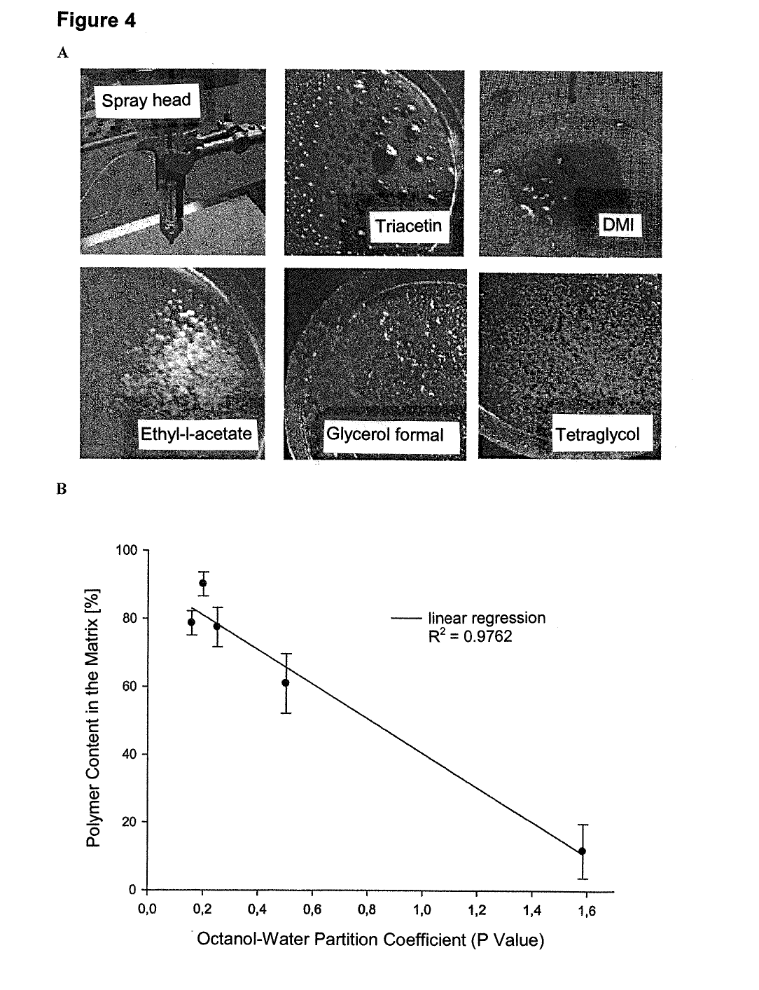

It has been found that the film quality of the film formed by the application system of the present invention is the higher, the higher the water miscibility of the solvent used. The Examples disclose tests for determining the film quality. FIG. 4 shows the film quality of a number of combinations of PLGA polymer and solvent. The water solubility of the above-mentioned solvents decreases in the following order: glycerol formal>DMI>tetraglycol. Thus, in most cases glycerol formal will be the most preferred solvent for the application system of the present invention, as long as it is capable of dissolving enough polymer. The following Table 2 provides an overview of the properties of some of the tested solvents:

TABLE 2
Properties of some Solvents

| | | | PLGA | | |
|---|---|---|---|---|---|
| Desig- | | | solu- | Appli- | |
| nation | η [mPas]* | XlogP3** | bility*** | cation | FAM |
| Glycerol | 14.4 | −0.8 | 33% | parenteral | Ivomec ® |
| formal | | | | | Peteha ® |
| Tetra- | 16.6 | −0.3 | n.s. | parenteral | Phenhydan ® |
| glycol | | | | | Eusaprim |
| DMI | 8.16 | −0.6 | 41% | topical | Mykosert ® |
| | | | | | Ibutop |
| | | | | | Gel ® |
| Triacetin | 18.75 | 0.2 | 35% | parenteral | n.s. |
| Ethyl | 2.74 | 0.2 | 46% | n.s. | n.s. |
| l-lactate | | | | | |

*The kinematic viscosity η was determined with a rotational viscometer (MRC 100, Paar Physics) at 25° C.,
**XlogP3 data are calculated values [32]
***Solubility data have been taken from a publication by Matschke et al., 2002 [33].

The film thickness of the matrix formed by the spray system of the invention plays a role for the diffusion rate of the water. For example, for PLGA systems with a film thickness of from 150 to 300 μm, in which the diffusion rate of the water is limited, surface erosion could additionally be detected. Independently of the solvent used, in the viscoelastic tests film layers of about 300 μm were measured for Resomer® RG 502 H-based films. By contrast, however, the film thickness of films for the longer-chain polymer increased analogous to the observed release kinetics from DMI via glyercol formal to tetraglycol.

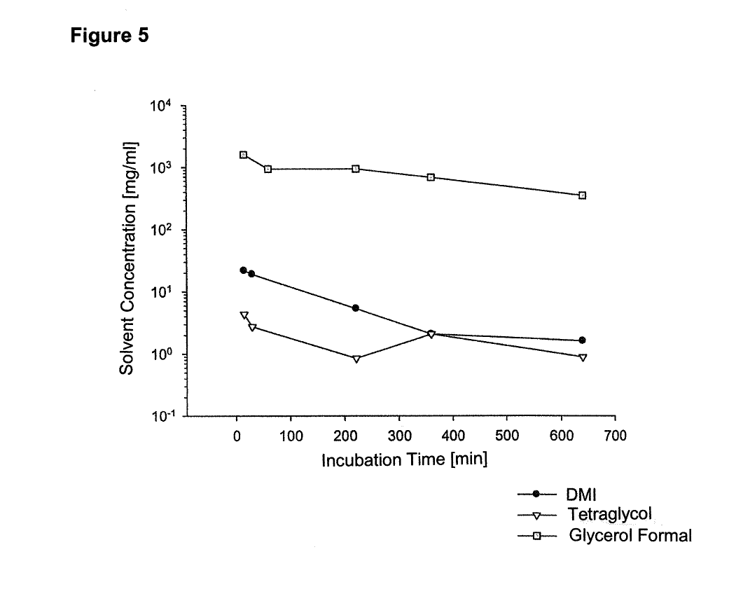

A further very important feature for the solvent to be used in the application system of the invention is its biocompatibility or tissue tolerance. In the present application, tissue tolerance is determined by the effect of the solvent on the metabolic cell viability over a period of 11 hours. A determination method is described in the Examples. The LD₅₀ value found therewith is the measure of toxicity. The LD₅₀ value must be at least 1, preferably at least 10 mg/ml, for a solvent to come into consideration for the present application system. The above-mentioned particularly preferred solvents fulfil this requirement. In this connection, glycerol formal has been found to be particularly suitable. It has an LD₅₀ value of about 1 g/ml at an incubation time of under 6 hours. Thus, glycerol formal represents a particularly preferred solvent for the system of the present invention. FIG. 5 shows LD₅₀ values of preferred solvents as function of incubation time.

In one embodiment, the application system of the present invention only comprises one liphophilic component with polymer and solvent, as described above, and water as second component. Provided they fulfil the above-mentioned requirements, it is possible with these components to produce, by mixing and spraying, a film in situ that can effectively prevent surgical adhesions.

It is essential for the invention that the spray system of the invention contains the lipophilic component and the aqueous component separate from each other until spraying. The components may only be mixed at or directly before spraying or during spraying. It has been found that the addition of comparatively small amounts of water already leads to polymer precipitation. Premature precipitation could interfere with film formation and the spraying device might possibly also be obstructed by polymer deposition. Therefore, mixing should preferably occur directly during spraying, e.g. by feeding the respective amounts of both components into a mixing chamber and then directly spraying them therefrom during mixing. Thus, mixing and spraying should preferably occur substantially at the same time.

In a further embodiment, an application system is provided that additionally includes an active agent. Suitable active agents are all substances useful for the targeted application site. The application system of the present invention is especially useful for releasing nucleic acids, proteins and peptides. Thus, it is possible to directly release proteins and peptides as well as the nucleic acids encoding them or even a mixture thereof. It has been found that the application system of the present invention and the film resulting therefrom releases the nucleic acids in such a form that their subsequent expression is possible. Since the system of the present invention is provided for the prevention of adhesions, preferably fibrinolytic proteins and peptides and/or the corresponding nucleic acids encoding them are used as active agents.

The active agent may be present in one of the two components in the dissolved or the dispersed state. It has been found that too high an amount of aqueous phase may (negatively) affect the quality of the film formed. Thus, if an active agent is to be added whose water solubility is not high enough for producing highly concentrated solutions it is preferable to add the active agent in already precipitated form, e.g. in the dry form. Lyophilisates or polyplexes in small-sized solid form that are dispersable in the lipophilic component are particularly suitable. This has the further advantage that, in its solid form, the active agent has a higher storage stability.

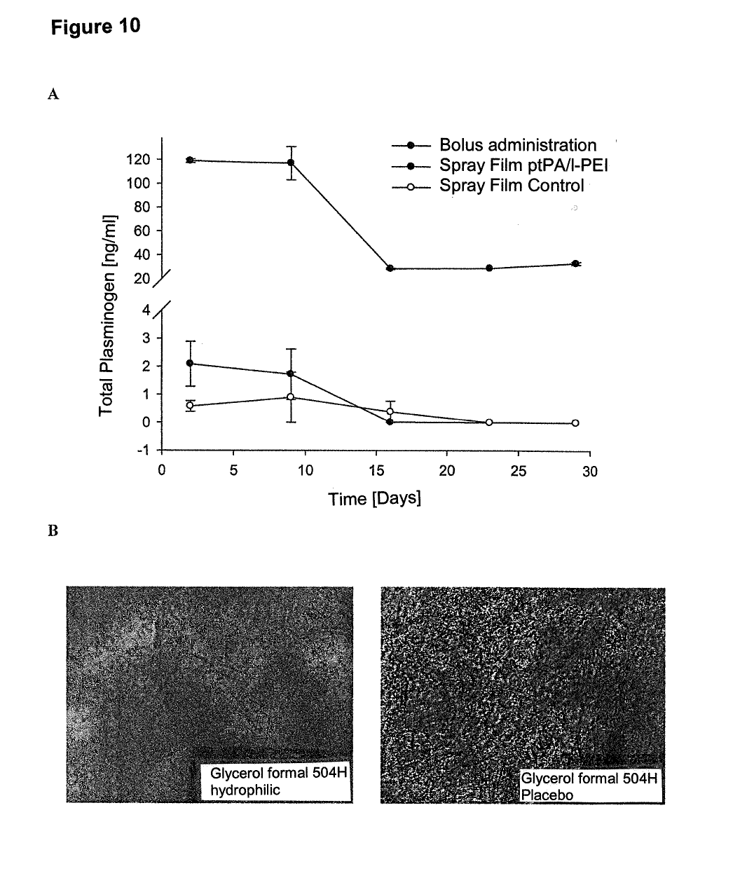

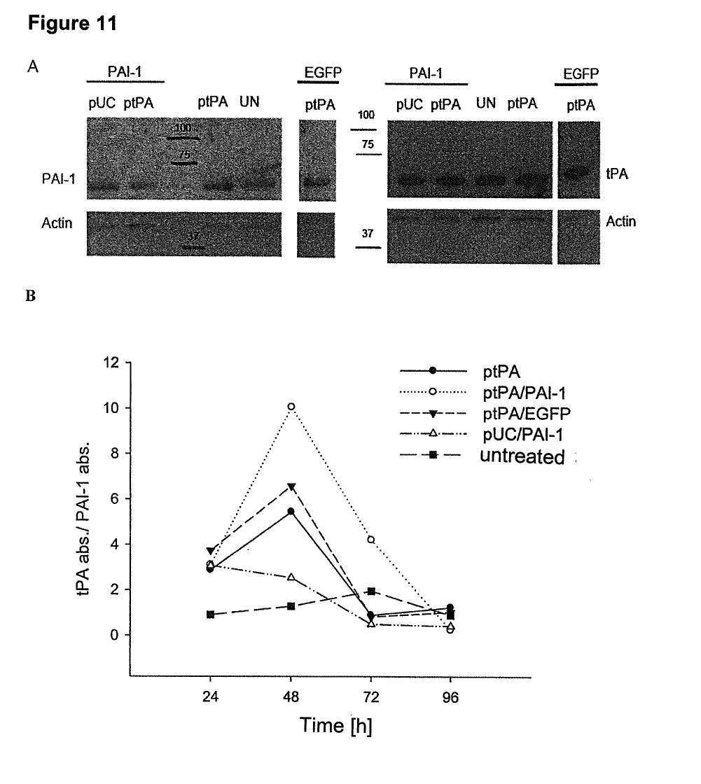

As stated above, especially tissue-specific plasminogen activators and their inhibitors play a role in the formation of adhesions. Thus, according to one embodiment of the invention a “gene activated” film formed in situ is locally applied by spraying on for the treatment of peritoneal adhesions. Since, as stated above, within a time slot of 2 to 3 weeks after surgery in the abdominal cavity, permanent adhesions may develop and since it is assumed that this is triggered by an imbalance between the tissue-specific plasminogen activator (tPA) and its inhibitor (PAI-1), this imbalance is changed in accordance with the present invention by providing tPA and/or inhibiting PAI-1. This is done with the film formed in situ which includes tPA and/or PAI-1 inhibitor and/or nucleic acids encoding them. It has been found that when a spray system of the present invention is used, which contains a plasmid coding for tPA, when a film is formed, the plasmid is incorporated into the film matrix, gradually released therefrom, and for at least two weeks raises the tPA level in the physiological environment. The tPA level in the physiological environment of the sprayed on film may also be raised by introducing a PAI-1 inhibitor into the environment or by a combination of both. In the Examples and FIGS. 10 and 11, the properties and results obtained with such films are described.

In this connection it was shown that incorporation of a tPA-encoding plasmid into a film of the invention based on glycerol formal with Resomer® RG 504 H in a cell culture assay could raise the tPA level for a period of 16 days to 2 ng/ml. This corresponds to a 4-fold increase of the tPA concentration compared to the control. Since in tissue that had been subject to surgery, and in inflamed tissue, the tPA concentration may drop to one fifth of the standard values and lower, it is thus possible with a film produced with the spray system of the invention to achieve, for an extended period of time, a therapeutically relevant increase of the tPA level, that could not be achieved with the prior art preparations.

It has furthermore been found that in stressed and/or inflamed tissue, the inhibitor level can be increased up to a factor of 17 which results in a significant lowering of the tPA level. Consequently, the tPA/PAI-1 ratio may vary from 3.5 in the normal state to 0.4 in inflamed tissue. Hence, to even more effectively control the processes occurring after surgery and to fight adhesions even more successfully, the spray system of the invention particularly preferably includes both at least one tissue-specific plasminogen activator or a nucleic acid coding therefor and at least one inhibitor of plasminogen activator inhibitor or a nucleic acid coding therefor. As shown in the Examples, the tPA/PAI-1 balance can efficiently be restored by producing, with the spray system of the invention, a film which causes a cotransfection of a tPA-encoding plasmid DNA and an siRNA against PAI-1. It could be shown that this cotransfection of a tPA-encoding plasmid DNA and an siRNA against PAI-1 leads 48 h after transfection to an 8.3 fold increase of the tPA/PAI-1 ratio, whereas the application of the plasmid alone will merely lead to an increase by the factor 4.5. Depending on the desired effect, the spray system of the invention may thus either include a tissue-specific plasminogen activator or at least one PAI-1 inhibitor or a combination of both and/or in each case the corresponding nucleic acids. Therefore, the system provided by the present invention allows a highly variable control of the desired effect.

In the above described embodiments, the nucleic acid may be RNA, DNA, mRNA, siRNA, miRNA, piRNA, shRNA, antisense-nucleic acid, aptamer, ribozyme, catalytic DNA and/or a mixture thereof. The term DNA comprises all suitable forms of DNA, such as cDNA, ssDNA, dsDNA, etc.; the term RNA comprises all suitable forms of RNA, such as mRNA, siRNA, miRNA, piRNA, shRNA, etc.

The nucleic acid may be linear or circular, it can be single stranded or double stranded. The term “nucleic acid” also covers a mixture of nucleic acids that can encode the same or different proteins or peptides. All forms of nucleic acids are suitable that encode the desired protein or peptide and are capable of expressing it at the desired site. The person skilled in the art knows the suitable forms of nucleic acids and is thus able to select the most suitable one. The nucleic acid may originate from any source, e.g. from a biological or synthetic source, from a gene library or a collection, it may be genomic or subgenomic DNA, RNA obtained from cells or microorganisms or synthetically produced RNA, etc. The nucleic acid may include the elements required for its amplification and expression, such as promotors, enhancers, signal sequences, ribosome binding sites, tails, etc.

The nucleic acid may be a DNA or RNA and it may comprise one or more genes or fragments. The nucleic acid may be an autonomously replicating sequence or integrating sequence, it may be present in the form of a plasmid, vector or another form well-known to the person skilled in the art. It may be linear or circular and single stranded or double stranded. Any nucleic acid active in a cell is suitable here. Since “naked” nucleic acids are not very stable and are rapidly inactivated or decomposed in the cell, it is preferable to coat the nucleic acid with a layer, with so-called polyplexes being a particularly preferred embodiment.

To protect the nucleic acid, it can be used in the form of so-called polyplexes. Polyplexes are nucleic acid molecules surrounded by a polymer envelope. Preferably, a cationic polymer is used as envelope material. It has been found that cationically charged particles can be more easily taken up by the cell than neutral or anionically charged particles. However, they may also promote more unspecific adsorptions. For enveloping nucleic acids, as active agents, cationic envelope materials are preferred, since nucleic acids can readily be enveloped and protected by cationic substances. Respective techniques are well-known to the person skilled in the art.

The envelope material may be a naturally occurring, synthetic or cationically derivatized natural substance, such as a lipid or a polymer or oligomer. An example of a natural oligomer is spermin. Examples of synthetic polymers are nitrogen-containing biodegradable polymers, especially those with protonable nitrogen atoms. Particularly suitable are polyethylene imines, in particular branched polyethylene imines, which are commercially available. Suitable is, for example, a branched polyethylene imine with a mean molecular weight of 25 kDa, which is commercially available. It has been found that this polymer is well compatible with the other components of the spray system of the present invention. It is also possible to use lipids, in particular cationic or neutral lipids, as natural or optionally derivatized film-forming envelope material. Lipids are available in many variants and may be used, for example, to form liposomes.

When polyplexes are used as active agent, the ratio of envelope material to nucleic acid should be adjusted in a manner known per se such that the nucleic acid is sufficiently protected but can still be expressed after release. If there is not enough envelope material, the nucleic acid will not be sufficiently protected. If the amount of the envelope material is too high, this may, on the one hand, lead to problems with tolerance, and, on the other hand, with too high an amount of envelope material, the nucleic acid may no longer be released and/or no longer be expressed. In both cases, the transfer efficiency is reduced. With a few routine tests, the person skilled in the art may find the best suitable ratio for the specific case. It has been found that a ratio of envelope material to nucleic acid in the range of from 10:1 to 1:4, based on the weight, is especially suitable. Particularly preferred is a ratio of envelope material to nucleic acid of from 4:1 to 1:4. When the polyplexes contain polyethylene imine as polymer, the polymer content may also be indicated by the molar ratio of polymer-nitrogen content to DNA-phosphate content (N/P); preferably the NP ratio is in a range of from 1 to 10, particularly preferably of from 4 to 8.

The polyplex molecules are designed such that the nucleic acid is protected during storage, transport, and until application, and that the nucleic acid is released and expressed at the target site. In the literature, suitable polymers have been described on many occasions and the person skilled in the art can select the most suitable one from a large number of materials.

Since the first clinical study in the year 1989, experience with nucleic acids as pharmaceutical substances has been gained in more than 1400 clinical studies. In addition to retroviral gene transfer systems mainly adenoviral ones were used, a large advantage being the efficiency of these systems. Thus, it could be shown that already the binding of a single virus particle is sufficient to infect the target cell. With regard to immunogenicity and potential mutagenesis, non-viral gene transfer systems are a safe alternative to viral systems. With reference to non-viral gene therapy approaches, there have been described the application of naked nucleic acid in combination with physical methods, such as electroporation, as well as the use of nano-scale complexes with synthetic carrier systems, such as cationic polymers, which are also called polyplexes. Information on production and use of polyplexes may, for example, be found in the article by Godbey W T, Mikos A G, “Recent progress in gene delivery using non-viral transfer complexes”. (J Control Release, 2001, 72:115-125), and information on cationic liposomes (lipoplexes) in articles by Lee R J, Huang L. “Lipidic vector systems for gene transfer” (Crit. Rev Ther Drug Carrier Syst. 1997; 14:173-206) and by Simoes S, Filipe A, Faneca H, Mano M, Penacho N, Duzgunes N, et al. “Cationic liposomes for gene delivery” (Expert Opin Drug Deliv. 2005, 2:237-254). The complexation systems are based on the principle that under physiological conditions the positively charged nucleic acid and the negatively charged carrier material spontaneously accumulate to nano-scale particles (“self-assembly”).

The spray system of the present invention provides a new and promising approach to achieving long-lasting gene expression. Through the formation of a film in the form of a gene-activated depot system, whose local application may lead to a constant nucleic acid level in the area of application for a defined period of time, advantageous properties are achieved. Therefore, it is possible to reduce the dosing frequency and dose amounts, to prevent undesirable side effects, such as the transfection of other tissues, i.e. so-called “off-target effects”, to avoid unphysiological protein levels and burdening patients with nucleic acid and carrier material, and to improve acceptance by patients.

As stated above, it is assumed that the ratio of PA to PAI-1 inhibitor has an impact on the formation of surgical adhesions and scar formation. Thus, in a further embodiment a spray system is provided which comprises a combination of PA and PAI-1 inhibitor and/or nucleid acids encoding them as active agents. Here, the ratio of PA to PAI-1 inhibitor is in the range of from 5:1 to 1:5; when the corresponding nucleic acids are used it is possible to set the ratio such that, after expression, a ratio of PA:PAI-1 inhibitor of from 5:1 to 1:5 is found at the target site. It has been found that when applying such a combination it is possible to particularly effectively suppress formation of surgical adhesions.

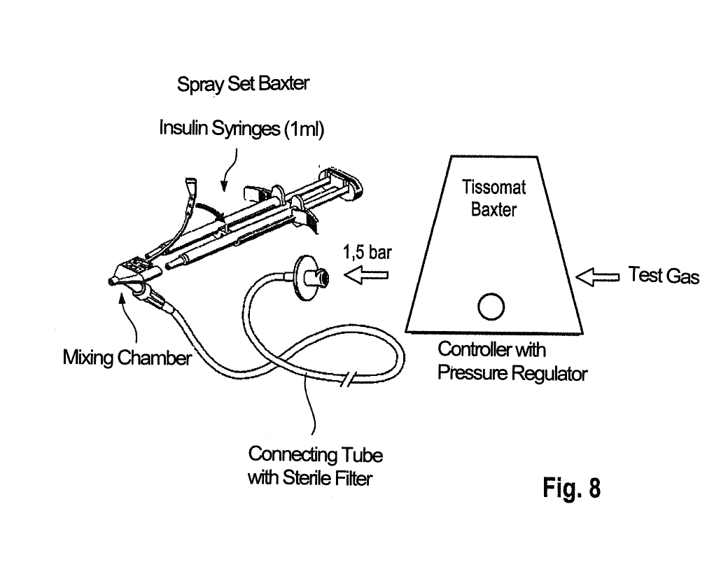

The spray system of the invention is characterized in that, upon mixing of the two components, the polymer is very quickly precipitated forming a film, with active agents optionally contained in one or both components being simultaneously co-integrated into the film. For this purpose, the two components, which prior to use are stored in separate containers, are sprayed in such a way that they are mixed at spraying or directly before spraying or that they are sprayed while being mixed. Thus, the two components of the spray system of the invention are mixed for application. Preferably, the two separate components are fed into a mixing chamber for spraying and are sprayed directly therefrom. Preferably used for spraying is a device known per se, wherein, upon activation of the spray valve, one dose each is fed into a mixing chamber from two repositories, and from there sprayed together. In this way, the mixing occurs directly in the spray applicator upon spraying, thus preventing a premature precipitation by which the spray nozzle could be clogged. With successive spraying of the two components from separate spray applicators it is not possible to produce a high-quality film. It is essential for the invention that the two components, which prior to their application have been kept separate from each other, come into contact with each other during spraying, so that, upon impact of the spray mist, the film formed by precipitation of the polymer can settle at the target site. Spray applicators suitable for the mixing/spraying of two components previously kept separately are known in the prior art. A known device suitable for the application in accordance with the present invention is shown in FIG. 8 and available as spray set from the company Baxter.

It is possible to control the dose amounts of the two components to be supplied. The respective dose amounts depend on the kind of use, the type of components, and optionally the active agent. Upon application, the two components should be mixed in a ratio (based on the volume of the solutions/liquids) of from 10:90 to 90:10, preferably of from 25:75 to 75:25, and more preferably in a ratio of from 40:60 to 60:40. The amount of the components supplied for generating the film depends on the desired size and thickness of the film. It may be adjusted in a manner known per se. For application in the abdominal cavity, a quantity of from 0.5 to 5 ml, preferably of from 0.7 to 3 ml of each component has been found to be suitable.

The following combinations have been found to be particularly advantageous:

lipophilic component: 10% (m/v) PLGA solution (Resomer® RG H series) in glycerol formal, tetraglycol or DMI,
hydrophilic component: water for injection,
active agent: pDNA/l-PEI polyplexes, as lyophilisate dissolved in the hydrophilic phase (incorporation option A) or by means of homogenizer dispersed in the lipophilic PLGA solution (incorporation option B).
optionally, sucrose in a concentration of 10% (m/v) as cryoprotector for lyophilization

The spray system of the present invention is provided for therapeutic application. The field of application for matrix systems generated therewith is the prevention of post-operative adhesions, which after surgery in the abdominal cavity may develop into permanent adhesions, caused by an imbalance between the tissue-specific plasminogen activator and its inhibitor [56, 64, 65]. Critically here is a time frame of 2 weeks comprising an acute phase of 2 to 5 days after surgery. Depot systems containing a tPA-encoding plasmid as active agent are particularly suitable. Beside the pharmacologically active component, the polymer film constitutes an additional anti-adhesive barrier against adhesions. For example, it is also possible to spray the spray system of the invention via endoscope, for example in the case of endoscopic interventions in the abdominal cavity.

The invention is further illustrated by the attached Figures.

The Figures show embodiments of the inventions and results obtained therewith.

FIG. 1 shows a schematic diagram relating to the pathogenesis of surgical adhesions.

FIG. 2 shows diagrams showing the film qualities of selected Resomer® RG polymers in comparison: A) PLGA 50:50, H series, B) PLGA 75:25, S series. It shows the percentage of the used polymer that forms the spray film, while the rest, as soluble portion, is lost in the supernatant. The data are given as mean values±standard deviation (n=4). Statistically significant differences are marked with asterisks (P<0.05 (*), P<0.01 (**)).

FIG. 3 shows diagrams of the results of viscoelastic tests of the films: A) storage modulus (G′), B) loss modulus (G″) of the Resomer® RG H series with different solvents compared at a frequency of 1 Hz.

FIG. 4 shows the film quality achieved with the tested solvents: A) films formed in situ using Resomer® RG 503 H; B) film quality plotted against the partition coefficient P. The data are shown as mean value±standard deviation of n=4 preparations.

FIG. 5 shows LD₅₀ values of the tested solvents in comparison: LD₅₀ of the tested solvents as function of the incubation time on mesothelial cells. In each case, the metabolic cell viability was determined by means of an ATPlite Assay.

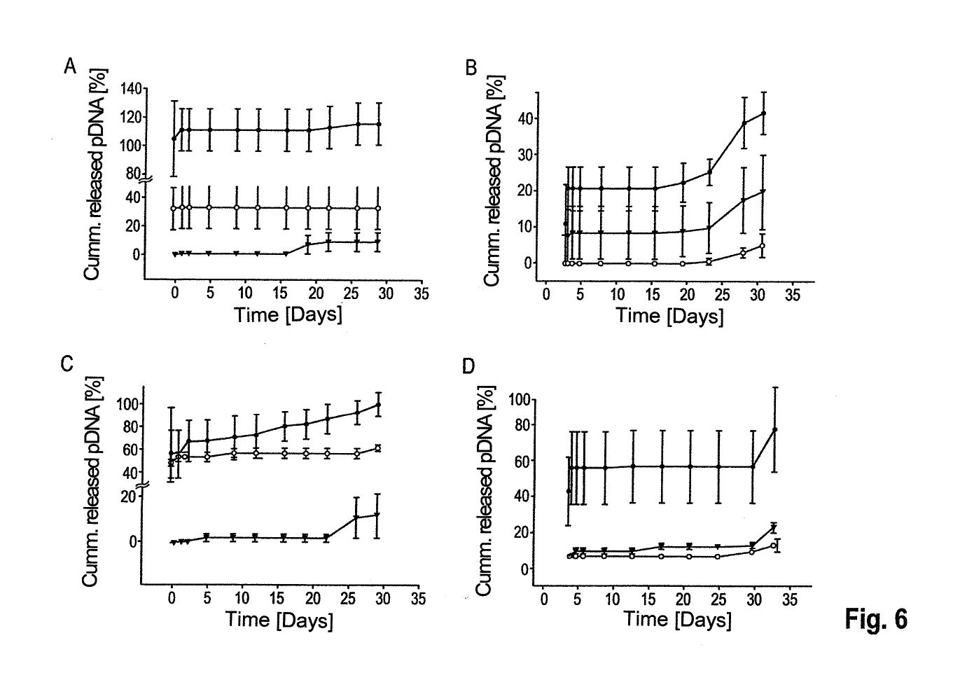

FIG. 6 shows diagrams on the release kinetics of different formulations of films formed in situ: (A) Resomer® RG 502 H, polyplexes in hydrophilic phase; (B) Resomer® RG 502 H, polyplexes in lipophilic phase; (C) Resomer® RG 504 H, polyplexes in hydrophilic phase; (D) Resomer® RG 504 H, polyplexes in lipophilic phase. Lyophilized polyplexes were incorporated into the films using different polymer solutions (DMI (), tetraglycol (◯) and glycerol formal (▾), and the release of the pDNA was analyzed for 30 days. The data are given as mean values±standard deviation from the mean value (n=3).

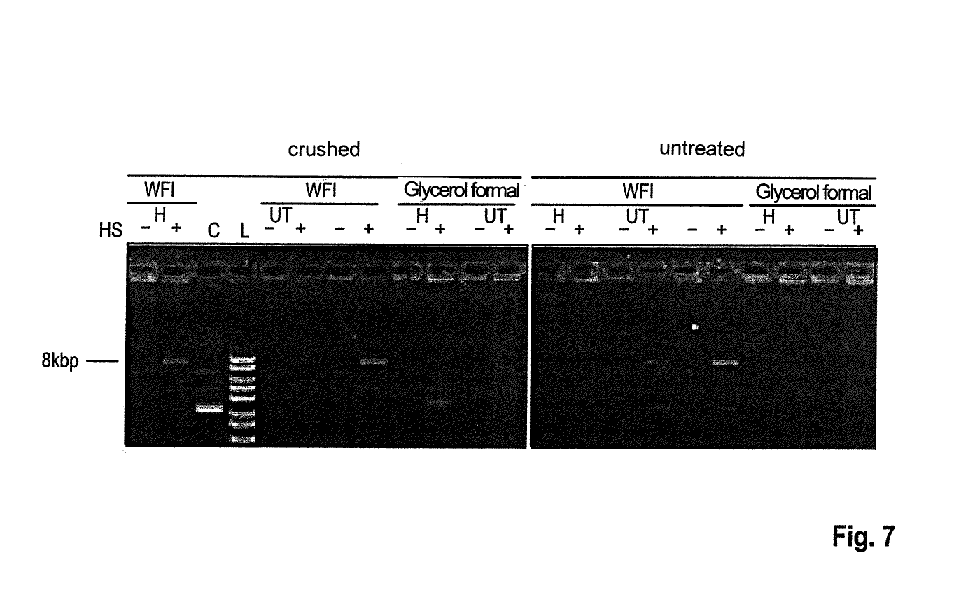

FIG. 7 shows the transfection efficiency of lyophilized l-PEI/pDNA polyplexes on lung cell lines using different cryoprotectors. DNA topology-lyophilized pDNA/l-PEI polyplexes were separated by agarose gel electrophoresis under addition of heparan sulfate (HS). For this purpose, the polyplexes were resuspended in water for injection (WfI). The data are shown as mean values±standard deviation ((a) n=7, (b) n=4)). Statistically significant differences are marked by asterisks (P<0.05 (*), P<0.01 (**)). pCMV-Luc control (C), size marker (L), water for injection (WfI), Ultra-Turrax® (UT), homogenizer (H).

FIG. 8 shows an experimental setup for the production of films formed in situ.

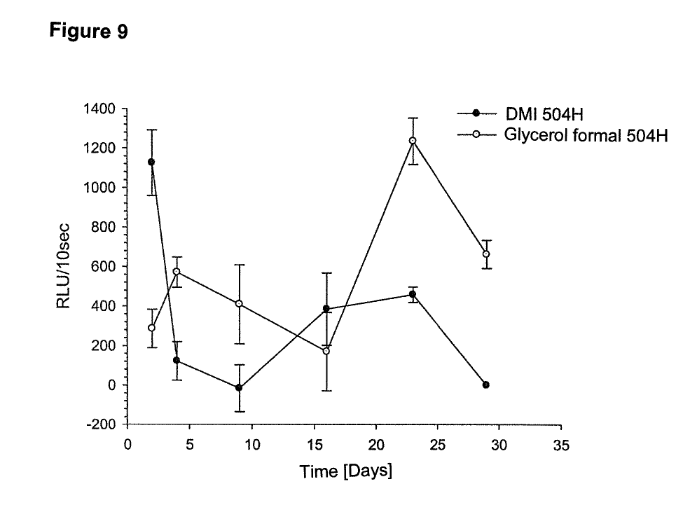

FIG. 9 shows a diagram with results of the in vitro application of films of the present invention on mesothelial cells: luciferase gene expression after application of Resomer® RG 504 H-based films on mesothelial cells. Plasmid DNA/l-PEI polyplexes were incorporated into the hydrophilic phase and their expression was studied over a period of 29 days by means of a luciferase assay. The emitted photons (RLU) were measured for 10 s after background correction. The results are given as mean values±SEM (standard error of means) (n=3).

FIG. 10 shows results of in vitro application of in situ formed films on mesothelial cells. (A) matrix release from Resomer® RG 504 H-based films and (B) fluorescence recording of the incorporated plasmid-DNA after staining with propidium iodide. pCMV-tPA-IRES-Luc/l-PEI polyplexes were dissolved in the hydrophilic phase, the spray film was sprayed on mesothelial cells, and the tPA level was determined for a period of 29 days by means of ELISA. The results are given as mean values±SEM (n=3).

FIG. 11 shows co-transfection of plasmid DNA/siRNA on mesothelial cells: a) PAI-1 and tPA detection in Western Blot after 48 h, b) tPA/PAI-1 ratio as function of time. The polyplexes comprising pCMV-tPA-IRES-Luc (ptPA) or a control plasmid (pUC) and different siRNAs (PAI-1, EGFP) were prepared with l-PEI at an N/P ratio of 10 (based on the amount of pDNA) in HBS. For comparison, the expression of untreated cells (UN) is shown. The tPA- and PAI-1 levels in the supernatant were determined by Western Blot at different times.

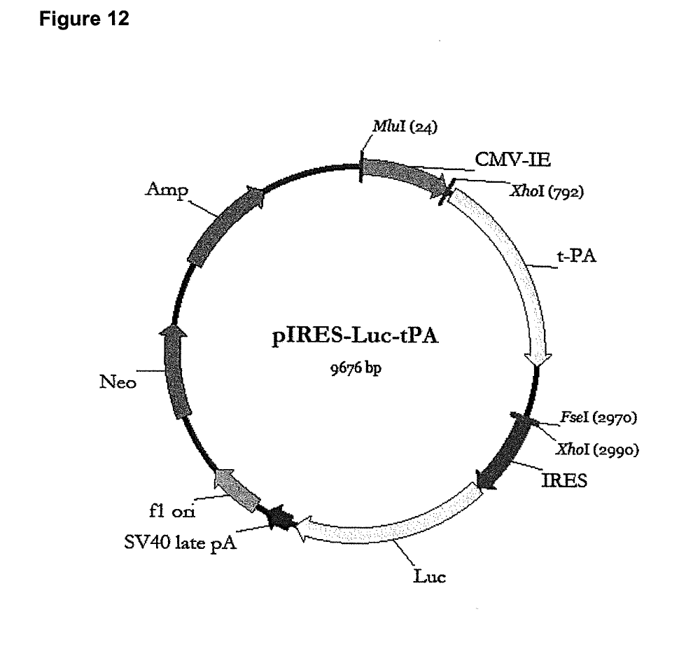

FIG. 12 shows a schematic diagram of the pCMV-tPA-IRES-Luc plasmid.

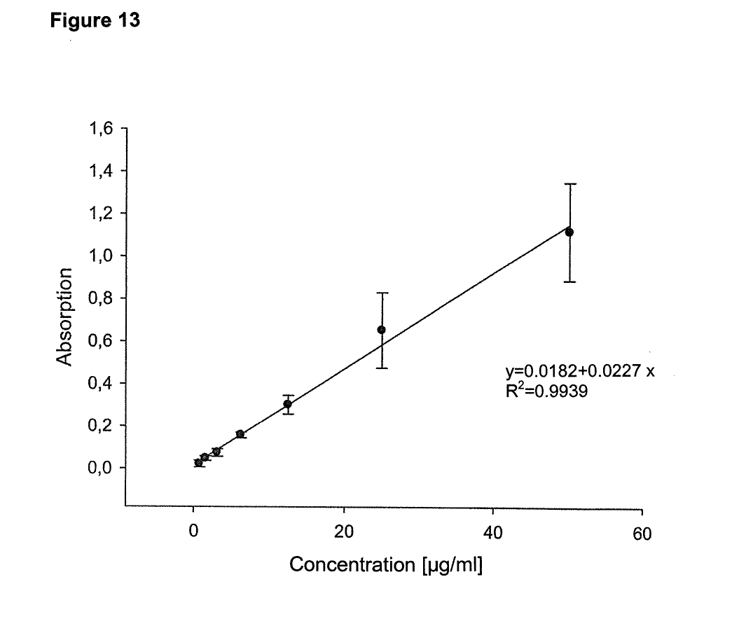

FIG. 13 shows a dilution series of l-PEI/pDNA polyplexes in PBS.

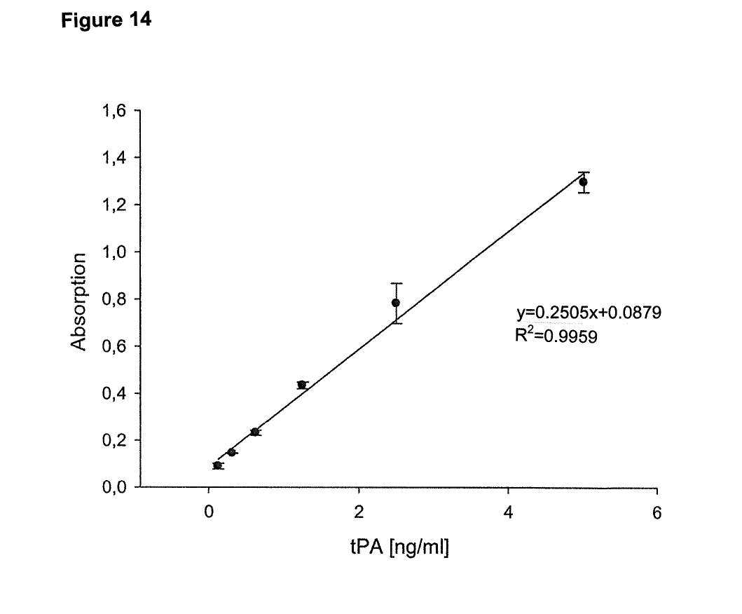

FIG. 14 shows a standard curve of the human tPA antigen assay.

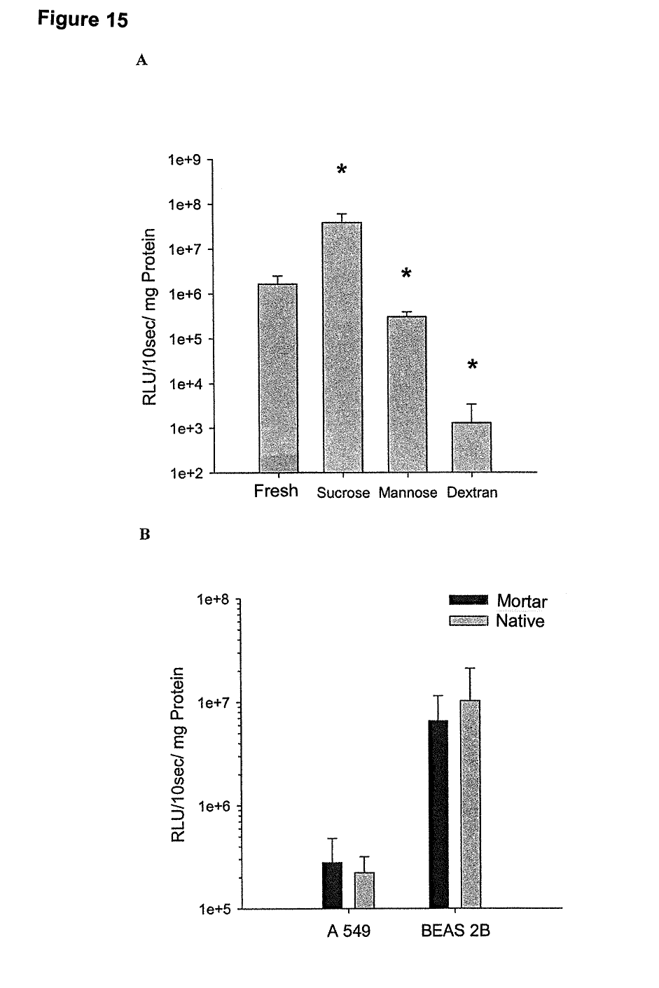

FIG. 15 shows the transfection efficiency of polyplexes in powder form using different cryoprotectors: transfection efficiency of lyophilized l-PEI/pDNA polyplexes on lung cell lines (A) using different cryoprotectors (10% (m/v) sucrose or mannose, 4% (m/v) dextran 5000, B) after homogenizaton of lyophilized polyplexes using 10% (m/v) sucrose as cryoprotector.

The invention is further illustrated by the following examples, without, however, restricting it in any way:

### EXAMPLE 1

### Materials and Methods

### Nucleic Acids

The plasmid pCMVLuc, obtainable as described in [19], contains the luciferase gene (Luc) of the firefly Photinus pyralis under the control of the CMV promoter, a promotor from the cytomegalo virus. Likewise under the control of the CMV promotor, the construct pMetLuc encodes the luciferase gene of the marine copepod Metridia longa, a secreted luciferase enzyme [20].

The construct pCMV-tPA-IRES-Luc was cloned and is schematically shown in FIG. 12. In addition to the sequences of the luciferase enzyme (Luc) and the tissue-specific plasminogen activator (tPA) it comprises a CMV promoter (CMV-IE, cytomegalo virus-immediate-early).

The pCMV-tPA-IRES-Luc plasmid was cloned using the pIRES-Luc vector [21]. A sequence coding for the tissue-specific plasminogen activator (tPA) was cloned into this vector under the control of the CMV promoter by using the restriction endonucleases MluI and FseI (New England Biolabs Inc., USA). For this purpose, the sequence (insert) of the plasmid pCMV-tPA was amplified by means of polymerase chain reaction (PCR) [22]. In addition to the luciferase gene, the pIRES-Luc vector contained an internal ribosomal entry site (IRES) which made it possible to translate both transcripts independently of each other.

The pUC21 vector (Invitrogen, Germany), which lacks an expression cassette and merely contains the bacterial backbone, was used as control plasmid.

The following oligonucleotides were synthesized: An siRNA was used against the plasminogen activator inhibitor 1 (PAI-1, 5′-GGAACAAGGAUGAGAUCAG[4, 23]-3′) and, as control, an siRNA against EGFP (5′-GCAAGCUGACCCUGAAGUUCAU[dT][dT]-3′). The lyophilized samples were dissolved in resuspension buffer (Qiagen) at 20 μM and for the release studies at 100 μM stock solutions, incubated for 1.5 min at 90° C., shaken gently for 1 h at 37 C, and stored in aliquots at −20° C.

### Polyethylenimine

Linear polyethylenimine having a molar mass of 22 kDa was synthesized according to a prescription by Plank et al. [24]. Analogous to the prescription, linear PEI was obtained by acidic hyrolysis of the proponic acid amide poly(2-ethyl-2-oxazoline) 50 Da, with the released propionic acid continuously being withdrawn from the synthesis batch as azeotropic mixture so that the reaction could almost completely run its course. Subsequently, the free base was precipitated by means of sodium hydroxide at pH 12, washed and lyophilized. The lyophilized l-PEI was stored at 4° C. and, as required, dissolved in distilled water, adjusted to a pH value of 7.4, dialyzed (ZelluTrans dialysis membranes T2, MWCO 8-10 kDa) and subjected to sterile filtration.

The PEI solution was quantified photometrically using the copper sulphate test at 285 nm on a spectrophotometer (Ultrospec 3100 Pro) [25]. An l-PEI batch of known concentration was used as reference. The purity of the synthesis product was checked by means of ¹H-NMR spectroscopy (Bruker 250 MHz, Karlsruhe). The molar mass was measured by means of gel permeation chromatography with a multi-angle laser light scattering detector (GPC-MALLS) and showed a molar mass of 20-22 kDa.

### PLGA Polymers

The following polymers from the company Boehringer Ingelheim, Germany, were used for the preparation of the films:
Resomer® RG 502, 503 and 504 H
Resomer® RG 752, 755 S

### Cell Line

Pleural mesothelial cells (human), in short Met5A, of ATCC, Germany (CRL-9444) were used. The cell line was cultivated in a 1:2 mixture of M199 (Gibco-BRL, Great Britain) and MCDB 105 (Sigma-Aldrich, Germany) at 37° C., 5% CO₂ and 100% humidity. In addition to 10% fetal calf serum (PPA Laboratories, Austria) an epidermal growth factor (5 ng/ml, Sigma-Aldrich, Germany) and hydrocortisone (400 ng/ml, Sigma-Aldrich, Germany) were added to the medium [26]. Furthermore, the cells were passaged at a confluence of about 80% and used for tests up to a passage of 20.

### Preparation of the Polyplexes

The formation of the complexes occurred spontaneously by electrostatic binding forces. The properties of the formed polyplexes substantially depended on the ionic strength of the medium, the polymers used and the N/P ratio. The latter specifies the molar ratio of protonated nitrogen atom (N) of the polymer structure to negatively charged phosphate atom (P) in the nucleic acid. To obtain small monodisperse particles, equal volumes of the solution with the lower charge density, the nucleic acid solution, were pipetted into the solution with higher charge density, the polymer solution, and mixed by adding and removing by pipette (5 to 8 times). Subsequently, the solution was incubated for 20 minutes at room temperature (RT) before further tests were made. Water for injection (siRNA, plasmid DNA lyophilisates) and HBS pH 7.4 (plasmid-DNA liquid) were used as medium.

### Preparation and Characterizaton of Polyplexes in Powder Form

The polyplexes were prepared using pCMVLuc and l-PEI at an N/P ratio of 10, as described above, in water for injection. To test different cryoprotective substances, the polyplexes, after the incubation period, were diluted with a 20% (m/v) sucrose solution, a 20% (m/v) mannose solution or a 4% (m/v) dextran 5,000 solution 1:2, mixed, and aliquoted. The aliquots could then be quick-frozen in nitrogen and lyophilized for about 24 h at maximum power in the freeze dryer. The lyophilisates were resuspended in the respective medium to a final concentration of 0.02 μg/μl (equal initial concentrations), and a transfection was made on BEAS-2B cells in 96-well plates, in an analogous manner as described below.

After an incubation time of 10 min, sucrose in powder form was added and the complexes were incubated for a further 10 min, with the particle size being controlled by PCS before and after addition of sucrose. After lyophilization, the powder could be homogenized in a mortar with pestle, and subsequently suspended in the PLGA solution with a homogenizer, a cylindrical glass vessel with glas pestle (Schütt Labortechnik, Germany), or by Ultra-Turrax® (level 3, 14 sec, Ika Labortechnik, Germany). Alternatively, the powder was either directly or after homogenization in a mortar resuspended in water for injection. The lyophilisates were resuspended in the respective medium to a final concentration of 0.02 μg/μl.

### Determination of Particle Size and Zeta Potential

The hydrodynamic cross section of the polyplexes was determined by photon correlation spectroscopy in a semi-micro cuvette with 600 μl polyplex solution in double-distilled water (0.02 μg/μl pDNA), the one of the Zeta potential by electrophoretic light scattering in a macro cuvette with 1.6 ml polyplex solution (0.02 and 0.1 μg/μl pDNA, respectively). The following settings were used: 5 measurements (size measurement), 5 runs à 10 cycles per sample (Zeta potential); viscosity of water (0.89 cP) and/or HBS (1.14 cP); refractive index 1.33; dielectric constant 78.5; temperature 25° C. The Zeta potential was calculated according to Smoluchowski. The evaluation of size was made on the basis of a standard curve. The apparatus was checked at regular intervals with polystyrene latex particles having a size of 92 nm (Duke Scientific Cooperation, CA, USA) and the Zeta potential reference Bl-LC-ZRZ with a charge of +50 mV (Laborchemie, Vienna, Austria).

### Agarose Gel Electrophoresis

With agarose gel electrophoresis it is possible to determine the degree of complexing of the nucleic acid (plasmid DNA, mRNA) in polyplexes. For this, polyplexes were prepared, as described above, combined with 6-fold concentrated loading buffer, and 100 ng pDNA each were applied to a 0.8% agarose gel containing ethidium bromide (10 μg/100 μl). A corresponding size marker was applied as reference. Electrophoresis was carried out at 125 V for about 1.5 h in 1×TAE buffer. Subsequently, the bands of the nucleic acid were detected under UV light (360 nm) and captured by gel camera.

In the agarose gel, an incomplete complexing can be recognized due to the presence of free nucleic acid in the gel, with polyplexes remaining in the gel pocket. Further, it is possible to draw conclusions with respect to the degree of condensation from the intensity of the signal in the gel pocket. The following applies: the weaker the band, the more nucleic acid is condensed by the cationic polymer. By the addition of a polyanion (0.1 μg heparan sulphate (HS)/μg pDNA, incubation time 45 min) the nucleic acid was displaced from the complex, and it became possible to check the integrity (topology, degradation) of the nucleic acid.

### EXAMPLE 2

### Characterization of Films Formed In Situ

### Determining the Film Quality as Function of Biomaterial and Solvent

To enable a further characterization of the film formation as function of the solvent used and the polymer type, spray tests were carried out in petri dishes. For this, a 10% (m/v) polymer solution was prepared in each solvent, and 1 ml polymer solution each (syringe 1) was sprayed with 1 ml water for injection (syringe 2) at 1.5 bar. A waiting time of 5 min was meant to allow complete formation of the matrix. For visual inspection of the matrix, the aqueous phase was stained with brilliant blue G and the distance to the spray surface was set at 11 cm. The further tests were made without fixation since it was thus possible to obtain a more homogenous film. The results are shown in FIG. 4.

To allow a better evaluation of the matrix quality, the supernatant was removed and dried in a vacuum system (Speed-Vac, Dieter Piatkowski, Germany) until constant weight. Analogously, the matrix was dried in a freeze dryer (Lyovac GT 2, LH Leybold, Germany) likewise until constant weight. It was then possible to determine the polymer content in the supernatant (loss) and in the precipitate (matrix quality) based on the total amount of polymer used.

### Determination of the Tissue Tolerance of the Solvent

The cytotoxicity of the solvent was determined by an ATP-based assay (ATPlite, Perkin Elmer). Cells were seeded into a 96-well plate 24 h before the assay, the medium was removed directly before the assay, the cells were washed once with PBS, and 50 μl of serum-containing medium with added antibotics (penicillin/streptomycin 0.1% (v/v); gentamycin 0.5% (v/v), Gibco-BRL, Great Britain) was added. Then, 50 μl each of the different solvent concentrations (16-500 μg/μl), diluted in water for injection, were added, and incubated at different lengths of time at 37° C., 5% CO₂ and 100% humidity (15, 30, 60, 221, 360 and 640 min). After the incubation period, the medium was sucked off, the cells were washed once with PBS, 50 μl PBS per well was added, and the cell viability was determined according to the manufacturer's instructions. The luminescence was measured in a plate reader (Wallac Victor2/1420 Multilabel Counter, PerkinElmer Inc., USA), with the luminescence of untreated cells (50 μl water for injection) being used as reference value with a viability of 100%. For each point in time, the concentration of the solvent was plotted against the measured cell viability (mean values±standard deviation from n=4 runs) and a non-linear standard function was adapted:

y=min+[(max−min)/(1+(x/EC50)^{Hilfslope})]

This function showed a good adjustment for all solvents: tetraglycol (R2=0.9181−0.9900), glycerol formal (R2=0.9268−0.9945), dimethyl isosorbide (R2=0.9647−0.9894). The LD₅₀ values were taken from the estimated values of the plotted regression curve. It is the concentration at which a cell viability of still 50% could be measured.

### Viscoelastic Properties of Films Formed In Situ

To get an idea of the viscoelastic properties of the matrix, rheological tests were carried out. For this purpose, the films were sprayed onto the plate of a rotational viscometer (Physica MCR 301) and the biomaterials (Resomer® RG 504 H and 502 H) were tested in a dynamic shear test in dependence on the solvent used. Here, a harmoniously oscillating shear stress with defined amplitude and frequency was applied to a sample and the resulting shear deformation was determined, which is characterized by two response parameters, the response amplitude and the response frequency, also called phase shift. Both response parameters can be mathematically converted into the storage modulus G′ and the loss modulus G″, with the storage modulus characterizing the stored and thus re-usable share of the introduced kinetic and/or deformation energy (elastic share) and the loss modulus being a measure of the energy given off in heat per oscillation and thus the lost share (frictional share).

### Tests for Determining the Release Kinetics

The tests for determining the release kinetics were carried out in lockable petri dishes (petri dishes without absorbent 50×9 mm, PAll) at 37° C. with continuous shaking in an incubator. For this purpose, the samples were sprayed as described above with water for injection. Lyophilized l-PEI/pCMVLuc complexes (N/P ratio 10, 10% sucrose, 25 μg pDNA/preparation) were previously dispersed in the PLGA solution in homogenized form (mortar and pestle) or resuspended in the aqueous phase. Water for injection was used as control. After spraying, it was waited for 5 min, the supernatant was removed (0 h value) and 1 ml PBS added. The supernatant was then completely exchanged at regular intervals, with the samples being stored at −20° C. until analysis.

The plasmid DNA released from the matrix formed in situ was quantified photometrically. For this purpose, the samples were extracted with chloroform prior to measuring (1 ml, 400 g, RT, 10 min) to separate PLGA degradation products that would interfer with photometric quantification [27]. The samples were subsequently photometrically measured at 260 nm (Nanodrop-1000, PEQLAB Biotech, Germany). In the run-up, l-PEI/pDNA polyplexes (pDNA concentration 100 μg/ml) were produced in water for injection and a standard series of serial dilutions with PBS was determined on 5 individual days at 260 nm, on the basis of which it was then possible to calculate the concentration of released complexed plasmid DNA. The results are shown in FIG. 13. As control, unloaded films were analyzed (background correction), small deviations in the volumes were taken into consideration by weighing the samples over the density of water.

### EXAMPLE 3

### In Vitro Analyses

### Transfections

For conducting in vitro transfection studies, 90,000 to 120,000 cells (24-well plate) per well were seeded 24 h prior to transfection. Only cells until passage 20 were used for transfection. This resulted in a confluence of about 70% on the day of transfection. Directly before transfection, the medium was removed, the cells were washed once with PBS and 200 μl (24 well plates) serum-free medium was added. Subsequently, 50 μl of the polyplexes, corresponding to a plasmid DNA amount of 1 μg (24 well plate), were added to the medium. After an incubation period of 4 h at 37° C., 5% CO₂ and 100% humidity, the polyplexes were removed, the cells were again washed once with PBS, and replaced by serum-containing medium with added antibiotics (penicillin/streptomycin 0.1% (v/v), gentamycin 0.5% (v/v), Gibco-BRL, Great Britain).

### Realization of the Spray Tests

L-PEI/plasmid DNA polyplexes (N/P ratio 10, 100 μg pDNA/preparation) were formulated, as described above, lyophilized with 10% sucrose and homogenized by means of mortar and pestle, so that they could be dosed by weight and either dispersed in a PLGA solution, which had previously been subjected to sterile filtration, or resuspended in the water phase (water for injection). Water for injection without additives was used as negative control. pMetLuc and pCMV-tPA-IRES-Luc were used in equal amounts as plasmid DNA.

3 days before the tests, Met5A cells were seeded onto hanging inserts (1 μm PET Millicell) with a polyethylene terephthalate-(PET) membrane, which allowed a control of the cells by light microscopy. 1.5 ml cell culture medium each was provided, the inserts equilibrated therein for 2 min, and subsequently 250,000 cells per well were seeded onto the membrane in 1.5 ml medium. Prior to the test, the medium was removed, washed once with PBS, and the samples were sprayed onto the cells, as described above. Initially, sampling was done daily, later every two to three days, and the medium was completely replaced. The samples were directly placed on ice and stored at −80° C. until analytical determination.

### Determination of the Transfection Efficiency by Means of Luciferase Activity Measurement

To analyse the gene transfer efficiency upon use of the pCMVLuc plasmid which codes for the reporter gene luciferase, the luciferase activity was measured 24 h after transfection by washing the cells once with PBS, adding 100 μl 1× cell lysis buffer (25 mM Tris/HCl pH 7.8, 0.01% Triton-X 100) per well, and, after an incubation time of 10 min at RT, shaking them for 60 sec. Subsequently, it was possible to automatically add 100 μA luciferin substrate (470 μM D-luciferin, 270 μM coenzyme, 33.3 mM DTT, 530 μM ATP, 1.07 mM (MgCO₃)₄Mg₂×5 H₂O, 2.67 mM MgSO₄, 0.1 mM EDTA 0.1 mM, 20 mM tricin) to an aliquot of 50 μl, and to measure the light emission for a period of 5 sec in a plate reader (Wallac Victor²/1420 Multilabel Counter, PerkinElmer Inc., USA). Before addition of the substrate, the background was likewise determined for a period of 5 sec. The luciferase activity, measured as emitted photons (Relative Light Units, RLU), was integrated after background correction for a period of 10 sec and based on the overall protein amount of the cell mass. The overall protein had previously been determined by means of a standard protein assay (method according to Biorad).

### Determination of the Transfection Efficiency Via Expression of Metridia Luciferase

First in vitro spray tests were conducted with a mixture of l-PEI/pMetLuc and l-PEI/pIRES-Luc-tPA polyplexes. The pMetLuc plasmid encodes a luciferase enzyme secreted by the cell, Metridia luciferase, and thus enables measuring of the gene transfer efficiency via the enzyme expression in the supernatant of the samples. This luciferase catalyzes the oxidative decarboxylation of the luciferin, in the present case of the coelenterazine, while at the same time emitting light at a wave length of 482 nm. At selected sampling times, samples were analyzed by means of a Ready-To-Glow Automation Kit (Clontech, A Takara Bio Company, France), by thawing them on ice and measuring the light emission for a period of 5 sec without prior dilution in accordance with the manufacturer's instructions in a plate reader (Wallac Victor²/1420 Multilabel Counter, PerkinElmer Inc., USA). Prior to addition of the substrate, the background was likewise determined for a period of 5 sec so that the luciferase activity (RLU values) could be integrated, after background correction, for a period of 10 sec, and the respective negative controls could be subtracted from the values. Untreated cells served as negative control for the bolus administration (single administration of the complete pDNA amount in water for injection) and unloaded films were used as negative control for the matrix systems.

### Determination of the Total Tissue Plasminogen Concentration by ELISA

In selected samples, in addition to the determination of the luciferase activity, the total tissue plasminogen concentration was determined by ELISA (Human tPA Total Antigenassays, Innovative Research, Dunn Labortechnik GmbH, Germany) in the supernatant of the cells. The used assay not only detected free and thus active tPA but also its latent form bound to the inhibitor. Since the supernatants were derived from the cell culture, the standard was diluted in an analogous manner as the samples in the cell culture medium of the used cells without FCS. The positive control (bolus administration) was diluted as follows: 1:50 (48 h, 9 d), 1:10 (16, 23 and 29 d). The samples from the inner compartment were filled up (30 μl sample ad 100 μl), while the samples from the outer compartment were analyzed without dilution. The assay was carried out in accordance with the manufacturer's instructions and the absorption at 450 nm was measured for a period of 0.1 sec in a plate reader (Wallac Victor²/1420 Multilabel Counter, PerkinElmer Inc., USA). The standard curve is shown in FIG. 14. The negative controls were used as described above.

### Fluorescence Micrographs

After completion of the spray test, the medium was removed and the plasmid DNA remaining in the matrix was stained with propidium iodide. For this purpose, the matrix was incubated with propidium iodide in a 1:10 dilution in PBS for 10 min at RT, again washed with PBS prior to picture taking, and pictures were taken with an epifluorescence microscope (Axiovert 135, Carl Zeiss, Jena, 10× lens). The excitation of propidium iodide occurred at 470±20 nm, while the emission was detected at 540±25 nm. The software Axiovision LE 4.5 was used for evaluation, and the analysis was done with an Alexa 560 nm filter at Brightfield.

### EXAMPLE 4

Cotransfection of siRNA and Plasmid DNA and Determination of the tPA/PAI-1 Ratio by Western Blot

Transfection was done as above in 24 well plates, with a few distinguishing features. In each case, 750 ng plasmid DNA and 30 pmol siRNA complexed with l-PEI at an N/P-ratio of 10 (based on the plasmid DNA amount) were used. The medium was changed after 6 h. After transfection, the proteins, i.e. the tissue-specific plasminogen activator and the type 1 plasminogen activator inhibitor (PAI-1), were analyzed by Western Blot. Since the proteins were secreted ones, they could be detected in the supernatant of the cells, so that a different points in time 20 μl each of the supernatant of the cells were removed, the samples per preparation (n=3) were pooled, and centrifugated at 14.000 rcf and 4° C. for 10 min to separate dead cells. The samples were consistently stored on ice and frozen at −20° C. until final analysis.

The proteins were separated in accordance with their molar mass by SDS-polyacrylamide gel electrophoresis (SDS-PAGE). To destroy secondary and tertiary structures of the proteins, the preparations (3.75 μl sample, 15 μl 4× loading buffer (130 mM Tris/HCl pH 7.4, 20% glycine, 10% SDS, 0.06% bromophenol blue, 4% DTT) ad 60 μl water for injection) had previously been denatured for 5 min at 95° C. 20 μl of each preparation were separated by electrophoresis on a 7.5% Tris-HCl gel (Bio-Rad Laboratories GmbH, Germany) and electrotransferred (1 h, 200 mA, transfer buffer) to a polyvinylidene difluoride (PVDF) membrane. After blotting, the membrane was cut, for incubation with various primary antibodies, at 50 Da (size standard, precision plus protein standards), and unspecific protein binding sites were blocked in a blocking buffer (5% milk powder in 20 mM Tris/HCl pH 7.4, 137 mM NaCl, 0.1% Tween20) for 1 h at RT with gentle shaking. The incubation with the primary antibodies took place overnight at 4° C. with gentle shaking (mouse anti-alpha-actin 1:15,000 Chemicon, Germany; mouse anti-huPAI-1 monoclonal 1:200 American Diagnostics, Germany; mouse anti-hutPA monoclonal 1:400 Calbiochem, Germany in 1:10 diluted blocking buffer). For detection, the secondary antibody (goat-anti-mouse HRP conjugated, Bio-Rad Laboratories, Germany) was used in a 1:10,000 dilution (tPA, PAI-1) or in a 1:20,000 dilution (actin), and the membrane was incubated for 1.5 h at RT with gentle shaking. Subsequently, the labelled proteins could be detected on a film (Amersham Hyperfilm ECL, GE Healthcare, Germany) by means of ECL chemiluminescence (Amersham Bioscience, USA) and were subjected to quantitation analysis by Image J Basics Version 1.38. The values were normalized based on the actin band of the untreated cells.

### Statistical Evaluation

Unless otherwise stated, the results are given as mean values±standard deviations. Statistically significant differences were calculated with the help of an unpaired t test. Statistical significance was assumed at α=0.05(*) or α=0.01(**), respectively.

The relevant parameters for the selection of a suitable solvent were i) pharmaceutical appliability, ii) good tissue tolerance, iii) water miscibility, and iv) solubility of the polymer in the solvent. Based on these parameters, some solvents were selected for a further screening.

A widely used solvent for the application of injectable depot systems is tetraglycol (tetrahydrofurfuryl alcohol polyethyleneglycol), also called glycofurol [28, 29]. Already since the sixties, tetraglycol has been used as solvent for parenterals (i.v., i.m.) in concentrations of up to 50% (v/v), and in this dilution only shows a low toxicity [30].

Glycerol formal is an odorless solvent with likewise low toxicity, consisting of a mixture of 1,3-dioxan-5-ol and 1,3-dioxolan-4-methanol [30]. It is an excellent solvent for numerous pharmaceuticals and cosmetics. These days it is mainly used in veterinary medicine as solvent for injections. For example, Ivomec™ 0.27% is approved for subcutaneous application in pigs and is used at 0.1 ml/kg [31].

With respect to dimethyl isosorbide (DMI) and ethyl lactate hardly any studies exist on the compatibility of the solvents. Ethyl lactate is used as parenterally applicable vehicle for steroid formulations and, in spite of its GRAS number, is considered to be relatively toxic with narcotic and mildly hemolytic activity. DMI is mainly topically used as penetration enhancer, for which a slight hemolytic activity was likewise observed [30, 34]. Furthermore, it could be shown in a study by Matschke et al. that glyerol esters have a good tolerability and are suitable solvents for PLGA/PLA polymers [33]. In this connection, triacetin was tested, a short chain triglyceride with low toxicity [35, 36], which already before had been described as alternative to NMP and DMSO for extended release formulations formed in situ [29, 37-39].

### Determination of the Film Quality in Dependence on the Solvent Used

The essential prerequisite for the formation of an in situ formed depot system is the solubility of the polymer in the solvent. In the literature, solubilities of at least 10% (m/v) are presumed for in situ formed systems based on PLGA [40]. Solubility data in classical solvents, such as NMP and DMSO, but also in ethyl lactate, have already been collected for different PLGA polymers [41]. The respective studies showed that the solubility of the polymers decreased with increasing molar mass. Furthermore, the amount of water required for an in situ precipitation correlated with the solubility of the polymer in the solvent used. The better the solubility of the polymer in the solvent used, the more water was required for the formation of the depot matrix. In contrast, the required amount of water decreased with increasing polymer content.

First formulation and solubility tests were made using the model polymer Resomer® RG 503H. For better visualization, the aqueous phase was stained with the blue dye brilliant blue. For all tested solvents, a 10% (m/v) polymer solution could be prepared. However, the films, already visually, showed clear differences with regard to stability, homogeneity and incorporation of the aqueous phase into the matrix. The results are shown in FIG. 4. As can be seen, with triacetin as solvent it was not possible to achieve a homogenous film, rather, a clearly visible W/O emulsion was formed. All other solvents led to the formation of a film, with the incorporation of the aqueous phase varying considerably. Based on the staining of the matrix, the incorporation of the aqueous phase decreased in the following order: triacetin≦ethyl lactate<<tetraglycol<glycerol formal˜DMI.

If one compares in FIG. 4 the incorporation of the aqueous phase into the polymer matrix with the Log P values of the individual solvents, it becomes clear that there is a dependence of the film quality, which is characterized by a low loss of polymer in the supernatant, on the water solubility of the solvents used. It must be assumed that triacetin and ethyl lactate, as lipophilic solvents (X log P>0), have a lower water miscibility than the other solvents, whereby the poor film quality of the matrix can be explained. Therefore, these solvents will only be taken into consideration for solution of very lipophilic PLGA polymers. Possibly, a mixture of solvents is more suitable. While with the test setup using triacetin it was not possible to produce a spray film, ethyl lactate resulted in a rather inhomogenous, porous film structure into which only very little dye could be incorporated. In contrast, in situ films foamed when glycerol formal, DMI or tetraglycol were used. Therefore, if possible, solvents having an X log P value of smaller 0 are preferred.

To be able to better evaluate the film quality, the amount of polymer in the supernatant (loss) and in the precipitate (implant) was quantified in spray tests by backweighing of the dried matrix. When the partition coefficient P of the solvents is plotted against the matrix quality, as shown in FIG. 4B, the film quality is found to be a linear function of the water miscibility of the solvent. The graph clearly shows that the matrix formation could be improved with increasing water miscibility of the solvent. At a P value of <0.25 about 80% of the amount of the polymer used was incorporated into the matrix.

For the further tests, different polymers were tested in combination with the solvents glycerol formal, DMI and tetraglycol. Triacetin was not further studied due to poor film formation, and ethyl lactate was not further investigated because of instabilities and a porous, inhomogenous film structure and because of its strong hemolytic activity.

### Tissue Tolerance of the Solvents

In addition, the influence of the solvent on the metabolic cell viability was studied for a period of 11 hours. Metabolic cell viability is determined by the ATP value of the cells, which is a measure of viability. With acute toxicity of substances, the value drops rapidly and thus allows an appraisal of the tissue tolerance of the solvents. FIG. 5 shows the LD₅₀ values, calculated from the tests, for all tested solvents as function of incubation time.

The tolerance of the solvents decreased in the following order: glyercol formal>>DMI>tetraglycol. With an LD₅₀ value of approximately 1 g/ml, at an incubation time of less than 6 h, glycerol formal showed the lowest toxicity of the tested solvents. Compared to DMI and tetraglycol, this meant a tolerance that was higher by a factor of 220 and a factor of 400, respectively, at the end of the test.

### EXAMPLE 5

### Analysis of Biomaterials

The analyzed biomaterials were biodegradable copolymers of lactic acid and glycolic acid, poly(D,L-lactic-co-glycolic acid) (PLGA) polymers, from the company Boehringer Ingelheim (trade name Resomer RG®), which have already been approved by the FDA for parenteral application.

### Matrix Quality in Dependence on the Selected Polymer

As described above, the percentage of the polymer content that can be incorporated into the film varied in dependence on the water solubility of the solvent used. Similarly, the matrix quality was to be studied in more detail using various polymers. Since, with regard to the tested polymers, tetraglycol could not be evaporated, no data exist for this solvent.

FIG. 2 shows the results for polymers having a composition of (a) PLA/PGA 50:50 with free acid groups (H series) and (b) PLA/PGA 75:25 with esterified end groups (S series). Due to their higher lactic acid content and the esterified end groups, the latter are more lipophilic than the H series.

The graphs illustrate that with higher molar mass of the polymers in both series a larger amount of polymer could be incorporated into the matrix. This effect was significant for DMI in the H series and for both solvents in the S series. When glycerol formal and Resomer® RG 755 S were used, almost 100% of the amount of polymer used formed the matrix (97.6±0.6%).

A comparison of the polymer series in combination with the tested solvents showed that glycerol formal with the S series had a 20% lower loss of the amount of polymer used compared with the H series and compared with DMI in both polymer series. These results can again be explained by the different water miscibility of the two solvents which led to a different solubility of the polymers in the solvent and had a significant influence on the kinetics of film formation. While both series were readily dissolvable in DMI, it was difficult to dissolve the S series in glycerol formal, compared to the H series. The latter showed strong swelling behavior. Thus, it must be assumed that the S series in glycerol formal constituted a system close to the solubility limit. In combination with the good water miscibility of glycerol formal, this led to rapid and complete film formation.

### Viscoelastic Properties of Films Formed In Situ

In a dynamic shear test, the viscoelastic properties of the films were analyzed. In this connection, an oscillating shear stress with a defined amplitude and frequency was applied to the sample, and the resulting shear deformation was determined, which is likewise characterized by amplitude and frequency, also called phase shift. These two response parameters can subsequently be mathematically converted into the storage modulus G′ and the loss modulus G″, with the storage modulus characterizing the stored and thus re-usable share of the introduced kinetic or deformation energy (elastic share) and the loss modulus being a measure of the energy given off in heat per oscillation and thus constituting the lost share (viscous share). In FIG. 3, both moduli are shown at an excitation frequency of 1 Hz for different films of the H series.

When comparing the elastic and the viscous shares of both films with each other, one recognizes a different sensitivity of the viscoelastic properties vis-à-vis the different solvents. While in case of the 502 H films, the viscoelastic properties could be adjusted quite broadly by selecting the suitable solvent (maximum factor: 29 (G′) and 22 (G″), respectively), the 504 H films showed a rheological behaviour independently of the solvent used. The latter showed with from 2 to 4 kPa a mechanical strength comparable to muscle fibres (8 to 17 kPa) [46] and were with a loss factor of (G′/G″)>1 mainly elastically dominated so that in the test these films behaved similar to a solid body. The only exception was DMI, whose in situ films were viscously dominated with a loss factor of 0.85. With a thickness of >500 μm, these films were relatively thick compared to the Resomer® RG 502 H films (DMI: 500 μm, glycerol formal: 600 μm, tetraglycol: 800 μm). The latter spread on the plate of the rheometer and showed a thickness of merely 300 μm independently of the solvent used.

All Resomer® RG 502 H-based films had a loss factor<1 and showed gel-like behavior, with significant differences in strength between the solvents being apparent. Merely, with tetraglycol a film strength comparable to that of Resomer® RG 504 H could be achieved at a loss factor of 0.79. With a strength of 130 and 900 Pa, respectively, DMI and glycerol formal were not even roughly comparable and showed a considerably viscously dominated behavior (loss factor>0.5).

### EXAMPLE 6

Formulation of Films Formed In Situ

Based on the experience from the already performed spray tests, a formulation was developed comprising i) PLGA polymer, dissolved in one of the three solvents already used, ii) aqueous phase, and iii) plasmid DNA as model active agent. Theoretically, the plasmid DNA could be incorporated into the matrix both in “naked” and in complexed form. Since, however, “naked” plasmid DNA transfected cells only rather inefficiently, the plasmid DNA was complexed with l-PEI (N/P ratio 10), prior to embedding into the film, and incorporated into the matrix as nano-scale polyplexes. By this, it could additionally be protected against a pH drop within the matrix, which occurs during degradation of the polymer structure through the release of polymer monomers within the matrix and generally constitutes a problem for sensitive macro molecules [47].

The polyplexes could be incorporated into the matrix either dissolved in the aqueous phase [44] or dispersed in the PLGA solution [28, 45]. However, it had been found in preliminary tests using PLGA/tetraglycol systems as example that already a direct addition of small amounts of water (about 5%) could induce a precipitation of the polymer. Therefore, with high loads of the spray film the use of highly concentrated plasmid DNA solutions was required, which, however, have low stability and tend to form aggregates. It was therefore advantageous to disperse the polyplexes as lyophilisate analogous to protein formulations [28, 45] in the PLGA solution or to resuspend them in the aqueous phase prior to use. Generally, the formulations were composed as follows:
1) lipophilic phase (1 ml): 10% (m/v) PLGA polymer in glycerol formal, tetraglycol or DMI (Resomer® RG 502 H, 504 H)
2) hydrophilic phase (1 ml): water for injection
3) active agent: lyophilized polyplexes resuspended in the lipophilic or hydrophilic phase

### Preparation of Polyplexes in Powder Form

In the pharmaceutical industry, lyophilization is one of the standard methods for stabilizing formulations during storage. By embedding the molecules into an adjuvant matrix, formulations can be stabilized during drying. Depending on the drying phase, different protectors may be used. Thus, cryoprotectors prevent crystallization of the solution during the freezing process. The system solidifies as undercooled melt without complete phase separation (solidified liquid, glass). In contrast, lyoprotectors provide protection in the further course of the freezing process. They replace the bonds of the active agent to water under formation of hydrogen bridges.

Polyplexes may also be lyophilized under addition of cryoprotectors and lyoprotectors, so that aggregate formation after resuspension can be prevented [48, 49]. As lyophilisate, polyplexes can be better stored [48] and a concentration of the solution up to a plasmid DNA concentration of 1 mg/ml becomes possible [50]. Sugars, such as sucrose or trehalose, act as lyoprotectors and cryoprotectors and have been found to be suitable for stabilizing polyplexes [48, 49]. Water-soluble substances like them may further accelerate the release of macromolecular active agents from PLGA-based films formed in situ. During matrix formation, water-filled pores develop as a result of dissolution of these substances, through which pores the active agent can subsequently diffuse from the matrix. A similar effect was described for a high load of the matrix [27, 33].

Various sugars were tested in concentrations used on a standard basis [48, 49]. The polyplexes, based on l-PEI, were resuspended in water for injection, after lyophilisation, without prior homogenization, and their transfection efficiency was tested on human bronchial epithelial cells. Good transfection rates were achieved in the concentrations used with the disaccharide sucrose with a 10-fold efficiency increase compared to freshly prepared polyplexes. The results are shown in FIG. 15. Comparable values were already described by Talsma et al. [48]. Osmotic effects that might lead to an increased incorporation of the particles into the cell could be expected only as from a 2-fold concentration onward [50]. However, particle size measurements showed that the particle size slightly increased after lyophilization. Freshly prepared particles had a particle size of ˜100 nm, while, depending on the dispersion medium used, the particles grew at a PI<0.2 to 200-300 nm after lyophilization.

To be able to dose the polyplexes which are in powder form and to incorporate them into the formulation, they should be homogenized in a mortar after lyophilization and dispersed in the PLGA solution by Ultra-Turrax (UT) or a glass homogenizer (H). The stability of the polyplexes after homogenization and subsequent resuspension in water for injection was analyzed by means of transfection tests and agarose gel electrophoresis. The results of the tests on lung cell lines showed no change in the transfection efficiency by homogenization (FIG. 15B). A control of the topology of the plasmid DNA under addition of heparan sulphate likewise failed to show a difference between the lyophilized polyplexes in water for injection (FIG. 7).

Both the samples resuspended in water for injection (untreated) and those polyplexes which had been homogenized (ground in the mortar) before resuspension showed similar band patterns for all three methods of dispersion (UT, homogenizer, dissolved). Compared to the control, uncomplexed plasmid DNA, in all cases a slight increase of the relaxed form was observed. For water for injection, a degradation of the plasmid DNA was not evident.

By way of example, glycerol formal is shown as solvent. No differences were found in the band patterns between untreated or prehomogenized samples and vis-à-vis the water control. Using the homogenizer as method of dispersion, no change could be observed either. With the Ultra-Turrax, the pDNA remained in the gel pockets mainly in complexed form. Here, for untreated samples two rather weak bands were detectable compared to the other samples. However, it should be noted that when the same amounts of heparan sulphate were used, under the influence of glycerol formal generally larger amounts of pDNA remained in the pockets in complexed form. With regard to the homogenized UT samples, a slight smear was seen in the gel; however, here again no destruction of the plasmid DNA in form of fragments could be observed.

### EXAMPLE 7

Determination of the Release Kinetics of Formulated Plasmid-DNA Polyplexes

The release of active agent from implants may in principle result from i) diffusion of the active agent from the polymer matrix (diffusion controlled) or ii) from erosion of the matrix (erosion controlled) [51, 52]. With respect to the release of active agents from bulk-eroding polymer matrices, as in the case of PLGA systems, one or two-phase release profiles are described in dependence on drug loading, molar mass of the polymer used, and polymer concentration [53, 54].

In addition, an initial release of the active agent may occur, even up to complete precipitation of the polymer. The release kinetics of films formed in situ were tested in dependence on the molar mass of the polymer, the solvent used, and the incorporation option. The following combinations were tested:
1. lipophilic phase (1 ml): Resomer® RG 502 H or 504 H 10% (m/v) dissolved in glycerol formal, tetraglycol or DMI
2. hydrophilic phase (1 ml): water for injection
3. active agent: lyophilized polyplexes (l-PEI/pDNA) in homogenized form

After lyophilization, the polyplexes were homogenized in a mortar and either resuspended in the hydrophilic phase (incorporation option A) or dispersed by homogenizer in the lipophilic PLGA solution (incorporation option B). The release profiles of both incorporation options using different solvents are shown in FIG. 6 for Resomers® RG 502 H and 504 H.

The diagrams show: Diagram (A): Resomer® RG 502 H, polyplexes in hydrophilic phase; diagram (B): Resomer® RG 502 H, polyplexes in lipophilic phase; diagram (C): Resomer® RG 504 H, polyplexes in hydrophilic phase, diagram (D): Resomer® RG 504 H, polyplexes in lipophilic phase.

When looking first at the incorporation of the polyplexes into the film by solution in the hydrophilic phase, one can clearly see a significant influence of the molar masses of the polymers used and of the solvents used on the release kinetics (FIGS. 6A, C). Using DMI as solvent in combination with the short-chain Polymer Resomer® RG 502 H, the active agent could not be incorporated (FIG. 6A). Already during precipitation of the polymer, 100% of the polyplexes were released. However, using the longer-chain polymer, the Resomer®RG 504 H, a typical two-phase release process was achieved. This comprised an initial diffusion-controlled release (phase 1, concave release profile) followed by an erosion-controlled release (phase 2, linear kinetics) (FIG. 6C). In this connection it was striking that when DMI was used in all cases the highest initial release was achieved, which varied of from 10% bis 100% in dependence on the incorporation option and chain length of the polymer.

In contrast, glycerol formal showed a low initial release, followed by slow diffusion-controlled release. Only with beginning erosion of the matrix erosion, an accelerated release of the polyplexes was observed, which depending on the chain length of the used polymer started after 15 and 26 days, respectively. Films based on tetraglycol, however, showed after a moderate initial release of 32% (Resomer® RG 502 H) and 50% (Resomer® RG 504 H), respectively, a moderate to zero release in the observed time frame. Merely in the case of the longer-chain polymers there was a low release after 26 days due to the erosion of the matrix (FIG. 6C).

However, when the polyplexes were dispersed in the lipophilic PLGA solution (incorporation option B), the initial release rates of the polyplexes and the diffusion from the matrix were reduced for all polymer/solvent combinations and the release proceeded mainly erosion-controlled (FIGS. 6B, D). The tested solvents showed similar release curves with differently strong retardation, with the release rates increasing in the following order: tetraglycol<<glycerol formal<DMI. While the release profiles of DMI and glycerol formal clearly showed shorter erosion rates for the short-chain 502 H polymer than for the long-chain 504 H polymer (day 17 versus day 29), in the case of tetraglycol no difference could be observed between the polymers (5.4% versus 8.8% cumulative release after 30 days) due to the strong retardation of the matrix.

In summary, DMI showed the fastest release for all combinations of long-chain or short-chain polymers and the various incorporation options. A continuous release of up to a 100% release of active agent could be achieved with Resomer® RG 504 H by incorporation of the active agent into the hydrophilic phase. Polyplexes which were incorporated into tetraglycol-based films showed no diffusion-controlled release. Over the observed period of time, after initial release, additionally up to 14% of the pDNA quantity could be released, with the initial release varying between 0 and 48%. In the case of incorporation option A it was comparatively high, while when the polyplexes were dispersed in the lipophilic phase, no initial release could be observed. Films on the basis of glycerol formal showed a low initial release, independently of the embedding method; however, even when these films were used, the polyplexes could only be released after 23 days by matrix erosion.

### EXAMPLE 8

Analysis of the Transfection Efficiency In Vitro

First in vitro release profiles were determined, as described above, by using the reporter gene luciferase. On the basis of these data and the requirements of the dosage form, the polymer Resomer® RG 504 H in combination with DMI as solvent was selected for the film. Both active agents were to be incorporated in lyophilized form into the hydrophilic phase of the matrix. From the preliminary tests, an initial release of active agent of 55% was expected. This could very well make sense for application in the field of postoperative adhesions to cover the acute phase after operation (days 2 to 5) [79-81]. However, here an unphysiological increase in the tPA concentration should be avoided because of an increased risk of bleeding in the peritoneum. Alternatively, a film without initial release, composed of Resomer® 504 H and glycerol formal, was tested. Here again, the polyplexes were dissolved in the hydrophilic phase. The formulations were first analyzed in vitro using active agent 1, which was complexed with l-PEI and lyophilized under addition of 10% sucrose. Additionally the pMetLuc plasmid encoding a luciferase enzyme secreted by the cell was used in a 1:1 mixture as control.

For in vitro testing in the cell assay, mesothelial cells (Met5A) were grown on inserts and the polymer film was subsequently sprayed onto the cell layer. The use of inserts enabled the partition of the wells into a two-chamber system with outer and inner compartment comparable to the anatomy in situ in the peritoneum, between which a constant exchange of substances was possible, so that the cells could be supplied with medium from the apical and the basolateral side. The cell morphology was optically controlled by light microscopy, which, however, was rendered difficult by the sprayed on film. The expression of the reporter gene luciferase could be analyzed by using the inserts over a period of 30 days in both compartments.

FIG. 10 shows the upper compartment. Compared to bolus administration, where the entire plasmid DNA dose was applied in water for injection without release system, the films formed in situ showed a luciferase level lower by a factor of 10³ to 10⁴. Gene expression proceeded as described above. In these studies on release kinetics, films on DMI basis showed an initial release of 56% of the pDNA quantity used, and, in the further course, showed a release of a further 38% until day 26. Following this release profile, for films on DMI basis an increased gene expression was observed already after 2 days, which after a further 7 days dropped to basic values, and until day 23 again increased to moderate values. In contrast, when glycerol formal was used as solvent, the gene expression occurred in two phases without an initial release of the polyplexes. After a first moderate increase of the expression rate in the first 10 days, a further maximum with a two-fold increased gene expression could be observed after 23 days. Low-molecular fragments in the cell culture medium indicated a beginning erosion.

The tPA expression from the matrix comprising glycerol formal and Resomer® RG 504 H is shown in FIG. 10A in comparison with a single dose and with a film without active agent (inactive film). Similar to the luciferase expression, a single dose of the active agent without depot system yielded extremely high protein levels over a period of 29 days, with a 100 to 40-fold increase of the tPA concentration compared to the basal values. Similar concentrations were achieved after intraperitoneal administration of recombinant tPA (alteplase) in plasma [82]. Therefore, the values that could be achieved by means of matrix formulation appear to be much closer to the physiological conditions. For plasma and peritoneal fluid, free tPA-antigen concentrations of from 4 to 6 ng/ml have been described [82-84], and additionally an average of 1.3 ng/ml tPA is bound to PAI-1 [84]. It is surprising that the values slightly increased already by application of the inactive films, with, at the beginning of the release, a 4-fold increase being achieved through the active film (with embedded active agent) compared to the inactive film. The effect leveled until day 16 and with 2 ng/ml the tPA levels dropped to basal values. This is presumably due to the fact that up to day 15 hardly any more polyplexes could be released from the matrix (see chapter 7.3). At the end of the tests, the matrix was not completely eroded, so that embedded polyplexes could be detected in the spray film (FIG. 10B).

### EXAMPLE 9

### Increase in the Tissue-Plasminogen Concentration Through Co-Application of siRNA and pDNA

An increase in the extracellular tPA concentration by application of a tPA-encoding plasmid could be successfully shown on mesothelial cells. In how far an increase in the tPA/PAI-1 ratio can be achieved by simultaneous application of an siRNA against PAI-1 will be analyzed in the following.

Previous studies had shown that l-PEI is mainly suitable for the in vivo application of siRNA and plasmid DNA [23]. Therefore, pDNA/siRNA/l-PEI polyplexes were prepared in HBS at an N/P ratio of 10 (based on the pDNA concentration), and different siRNA sequences against PAI-1 were tested. The tPA/PAI-1 ratio with different pDNA/siRNA combinations is shown in FIG. 11. The siRNA sequence used was the one which had most efficiently inhibited the PAI-1 expression in preliminary tests (PAI-1 A), at an optimized concentration of 0.12 μM. 48 h after transfection, the tPA/PAI-1 ratio with coapplication increased by the factor of 8, compared to untreated cells. Through application of pDNA alone or in combination with a non-functional siRNA (EGFP siRNA) merely a 4-fold and 5-fold increase, respectively, could be achieved. When a control plasmid (pUC) was used, it was found that the siRNA caused an increase of the tPA/PAI-1 ratio by a factor of 2.

### EXAMPLE 10

The development of a film formed in situ for the controlled release of polyplexes was made on the basis of an application system from the company Baxter, as shown in FIG. 8. For this purpose, the two-syringe system was loaded with a lipophilic component (syringe 1), comprising a biodegradable polymer dissolved in an organic solvent, and an aqueous component (syringe 2), water for injection. Lyophilized pDNA/l-PEI polyplexes were used as active agent. These could subsequently be dispersed in the lipophilic phase or dissolved in the hydrophilic phase. Both incorporation options were analyzed as above with respect to the release kinetics.

### EXAMPLE 11

In the cell viability test, glycerol formal showed the best compatibility on mesothelial cells. Here, LD₅₀ values higher by a factor of 200 and 400, respectively, were measured, compared to DMI and tetraglycol. At an incubation period of under 6 h, these lay by about 1 g/ml, i.e. when about 780 μl of pure glycerol formal were used, 50% of the mesothelial cells died. Literature data disclose comparable LD₅₀ values for DMI and glycerol formal after i.v. administration in rodents, while tetraglycol is more toxic by a factor of 2 to 3. From the manufacturer's information regarding Ivomec®, an antiparasitic veterinary drug, an LD₅₀ of from 4 to 4.8 g/kg body weight (mouse) upon i.v. application of a 50% glycerol formal solution can be derived. This is similar to what the EMA describes in a summary report of the Committee for Veterinary Products [85]. The acute toxicity after i.v. administration of DMI only minimally differs from glycerol formal. With an LD₅₀ of 5.4 g/kg body weight (rat) upon application of 40% DMI in isotonic saline solution (v/v) and an LD₅₀ of 6.9 g/kg body weight (mouse) upon application of a 20% solution, DMI seems after i.v. administration to be better tolerated. Already since the sixties, tetraglycol has been used in concentrations of up to 50% (v/v) as solvent for parenterals (i.v., i.m.), and in this dilution is classified as non-irritant. The LD₅₀ after i.v. administration without dilution is at 3.8 g/kg body weight (mouse) lower than described for the two other solvents [86].

### REFERENCES

[1] Rosenberg S A, Aebersold P, Cornetta K, Kasid A, Morgan R A, Moen R, et al. Gene transfer into humans—immunotherapy of patients with advanced melanoma, using tumor-infiltrating lymphocytes modified by retroviral gene transduction. N Engl J Med. 1990; 323:570-578.
[2] Edelstein M L, Abedi M R, Wixon J. Gene therapy clinical trials worldwide to 2007—an update. J Gene Med. 2007; 9:833-842.
[3] Gene Therapy Clincial Trials Worlwide. Journal of Gene Medicine; 2009.
[4] Seisenberger G, Ried M U, Endress T, Buning H, Hallek M, Brauchle C. Real-time single-molecule imaging of the infection pathway of an adeno-associated virus. Science. 2001; 294:1929-1932.
[5] Godbey W T, Mikos A G. Recent progress in gene delivery using non-viral transfer complexes. J Control Release. 2001; 72:115-125.
[6] Lee R J, Huang L. Lipidic vector systems for gene transfer. Crit. Rev Ther Drug Carrier Syst. 1997; 14:173-206.
[7] Simoes S, Filipe A, Faneca H, Mano M, Penacho N, Duzgunes N, et al. Cationic liposomes for gene delivery. Expert Opin Drug Deliv. 2005; 2:237-254.
[8] Nishikawa M, Takakura Y, Hashida M. Pharmacokinetics of Plasmid DNA-Based Non-viral Gene Medicine. Adv Genet. 2005; 53 PA:47-68.
[9] Pack D W, Hoffman A S, Pun S, Stayton P S. Design and development of polymers for gene delivery. Nat Rev Drug Discov. 2005; 4:581-593.
[10] Pannier A K, Shea L D. Controlled release systems for DNA delivery. Mol Ther. 2004; 10:19-26.
[11] Elfinger M, Uezguen S, Rudolph C. Nanocarriers for Gene Delivery—Polymer Structure, Targeting Ligands and Controlled-Release Devices. Current Nanoscience. 2008; 4:322-353.
[12] Packhaeuser C B, Schnieders J, Oster C G, Kissel T. In situ forming parenteral drug delivery systems: an overview. Eur J Pharm Biopharm. 2004; 58:445-455.
[13] Hatefi A, Amsden B. Biodegradable injectable in situ forming drug delivery systems. J Control Release. 2002; 80:9-28.
[14] Dadey E J. The Atrigel Drug Delivery System. In: Rathbone M J, Hadgraft J, Roberts M S, Lane M E, editors. Modified-release drug delivery technology. 2 ed. New York: Inform a Healthcare USA, Inc.; 2008. p. 183-190.
[15] Chen G, Junnarkar G. Alzamer Depot Bioerodible Polymer Technology. In: Rathbone M J, Hadgraft J, Roberts M S, Lane M E, editors. Modified-release drug delivery technology. 2 ed. New York: Informa Healthcare USA, Inc.; 2008. p. 215-225.
[16] Brodbeck K J, DesNoyer J R, McHugh A J. Phase inversion dynamics of PLGA solutions related to drug delivery. Part II. The role of solution thermodynamics and bath-side mass transfer. J Control Release. 1999; 62:333-344.
[17] Graham P D, Brodbeck K J, McHugh A J. Phase inversion dynamics of PLGA solutions related to drug delivery. J Control Release. 1999; 58:233-245.
[18] Fried I, Traitel T, Goldbart R, Shmilowitz D, Wolfson M, Kost J. Cancer gene therapy using pH-sensitive polyplexes released from an injectable PLGA implant. In: Society C R, editor. 33rd Annual Meeting of the Controlled Release Society Vienna, Austria: Controlled Release Society 2005. p. 598.
[19] Plank C, Zatloukal K, Cotten M, Mechtler K, Wagner E. Gene transfer into hepatocytes using asialoglycoprotein receptor mediated endocytosis of DNA complexed with an artificial tetra-antennary galactose ligand. Bioconjug Chem. 1992; 3:533-539.
[20] Markova S V, Golz S, Frank L A, Kalthof B, Vysotski E S. Cloning and expression of cDNA for a luciferase from the marine copepod Metridia longa. A novel secreted bioluminescent reporter enzyme. J Biol Chem. 2004; 279:3212-3217.
[21] Sakata Y, Yoshioka W, Tohyama C, Ohsako S. Internal genomic sequence of human CYP1A1 gene is involved in superinduction of dioxin-induced CYP1A1 transcription by cycloheximide. Biochem Biophys Res Commun. 2007; 355:687-692.
[22] Sakamoto T, Oshima Y, Nakagawa K, Ishibashi T, Inomata H, Sueishi K. Target gene transfer of tissue plasminogen activator to cornea by electric pulse inhibits intracameral fibrin formation and corneal cloudiness. Hum Gene Ther. 1999; 10:2551-2557.
[23] Bonnet M E, Erbacher P, Bolcato-Bellemin A L. Systemic delivery of DNA or siRNA mediated by linear polyethylenimine (L-PEI) does not induce an inflammatory response. Pharm Res. 2008; 25:2972-2982.
[24] Orgris M, Wagner E. Linear polyethylenimine: Synthesis and transfection procedures for in vitro and in vivo. In: Friedman T, Rossi J, editors. Gene Transfer: Delivery and Expression of cDNA and RNA, A Laboratory Manual: Cold Spring Habor Laboratory Press; 2007. p. 521-528.
[25] Ungaro F, De Rosa G, Miro A, Quaglia F. Spectrophotometric determination of polyethylenimine in the presence of an oligonucleotide for the characterization of controlled release formulations. J Pharm Biomed Anal. 2003; 31:143-149.
[26] Arbeiter K, Bidmon B, Endemann M, Bender T O, Eickelberg O, Ruffingshofer D, et al. Peritoneal dialysate fluid composition determines heat shock protein expression patterns in human mesothelial cells. Kidney Int. 2001; 60:1930-1937.
[27] Csaba N, Caamano P, Sanchez A, Dominguez F, Alonso M J. PLGA:poloxamer and PLGA:poloxamine blend nanoparticles: new carriers for gene delivery. Biomacromolecules. 2005; 6:271-278.
[28] Eliaz R E, Kost J. Characterization of a polymeric PLGA-injectable implant delivery system for the controlled release of proteins. J Biomed Mater Res. 2000; 50:388-396.
[29] Chern R Z, J. Liquid polymeric compositions for controlled release of bioactive agents. In: Patent U, editor. 2004.
[30] Mottu F, Laurent A, Rufenacht D A, Doelker E. Organic solvents for pharmaceutical parenterals and embolic liquids: a review of toxicity data. PDA J Pharm Sci Technol. 2000; 54:456-469.
[31] Lifschitz A, P is A, Alvarez L, Virkel G, Sanchez S, Sallovitz J, et al. Bioequivalence of ivermectin formulations in pigs and cattle. J Vet Pharmacol Ther. 1999; 22:27-34.
[32] Cheng T, Zhao Y, Li X, Lin F, Xu Y, Zhang X, et al. Computation of octanol-water partition coefficients by guiding an additive model with knowledge. J Chem Inf Model. 2007; 47:2140-2148.
[33] Matschke C, Isele U, van Hoogevest P, Fahr A. Sustained-release injectables formed in situ and their potential use for veterinary products. J Control Release. 2002; 85:1-15.
[34] Mottu F, Stelling M J, Rufenacht D A, Doelker E. Comparative hemolytic activity of undiluted organic water-miscible solvents for intravenous and intra-arterial injection. PDA Pharm Sci Technol. 2001; 55:16-23.
[35] Jain R A. The manufacturing techniques of various drug loaded biodegradable poly(lactide-co-glycolide) (PLGA) devices. Biomaterials. 2000; 21:2475-2490.
[36] Jain R A, Rhodes C T, Railkar A M, Malick A W, Shah N H. Controlled release of drugs from injectable in situ formed biodegradable PLGA microspheres: effect of various formulation variables. Eur J Pharm Biopharm. 2000; 50:257-262.
[37] Chandrashekar G, Udupa N. Biodegradable injectable implant systems for long term drug delivery using poly (lactic-co-glycolic) acid copolymers. J Pharm Pharmacol. 1996; 48:669-674.
[38] Singh U V, Udupa N. In vitro characterization of methotrexate loaded poly(lactic-co-glycolic) acid microspheres and antitumor efficacy in Sarcoma-180 mice bearing tumor. Pharm Acta Helv. 1997; 72:165-173.
[39] Bleiberg B, Beers T R, Persson M, Miles J M. Metabolism of triacetin-derived acetate in dogs. Am J Clin Nutr. 1993; 58:908-911.
[40] Dunn R, English J, Cowsar D, Vanderbilt D. Biodegradable in-situ forming implants and methods for producing the same. In: Patent U, editor. U S 1994.
[41] Shively M, Coonts B, Renner W, Southard J, Bennett A. Physico-chemical characterization of a polymeric injectable implant delivery system. J Control Release. 1995; 33:237-243.
[42] Sitter T, Toet K, Quax P, Kooistra T. Fibrinolytic activity of human mesothelial cells is counteracted by rapid uptake of tissue-type plasminogen activator. Kidney Int. 1999; 55:120-129.
[43] Elfinger M, Maucksch C, Rudolph C. Characterization of lactoferrin as a targeting ligand for nonviral gene delivery to airway epithelial cells. Biomaterials. 2007; 28:3448-3455.
[44] Eliaz R E, Szoka F C, Jr. Robust and prolonged gene expression from injectable polymeric implants. Gene Ther. 2002; 9:1230-1237.
[45] Eliaz R E, Wallach D, Kost J. Delivery of soluble tumor necrosis factor receptor from in-situ forming PLGA implants: in-vivo. Pharm Res. 2000; 17:1546-1550.
[46] Engler A J, Sen S, Sweeney H L, Discher D E. Matrix elasticity directs stem cell lineage specification. Cell. 2006; 126:677-689.
[47] Capan Y, Woo B H, Gebrekidan S, Ahmed S, DeLuca P P. Influence of formulation parameters on the characteristics of poly(D, L-lactide-co-glycolide) microspheres containing poly(L-lysine) complexed plasmid DNA. J Control Release. 1999; 60:279-286.
[48] Talsma H, Chemg J, Lehrmann H, Kursa M, Ogris M, Hennink W E, et al. Stabilization of gene delivery systems by freeze-drying. Int J Pharm. 1997; 157:233-238.
[49] Chemg J Y, vd Wetering P, Talsma H, Crommelin D J, Hennink W E. Stabilization of polymer-based gene delivery systems. Int J Pharm. 1999; 183:25-28.
[50] Anchordoquy T J, Armstrong T K, Molina M C. Low molecular weight dextrans stabilize nonviral vectors during lyophilization at low osmolalities: concentrating suspensions by rehydration to reduced volumes. J Pharm Sci. 2005; 94:1226-1236.
[51] Fan L, Singh S. Controlled release: a quantitative treatment 1989.
[52] Goepferich A. Mechanisms of polymer degradation and elimination. Handbook of biodegradable polymers 1997. p. 451-471.
[53] Goepferich A, Sieh L, Langer R. Aspects of polymer erosion. Materials Research Society Symposium Proceedings 1995. p. 155-160.
[54] Ramchandani M, Robinson D. In vitro and in vivo release of ciprofloxacin from plga 50:50 implants. J Control Release. 1998; 54:167-175.
[55] Menzies D. Peritoneal adhesions. Incidence, cause, and prevention. Surg Annu. 1992; 24 Pt 1:27-45.
[56] Hellebrekers B W, Trimbos-Kemper T C, Trimbos J B, Emeis J J, Kooistra T. Use of fibrinolytic agents in the prevention of postoperative adhesion formation. Fertil Steril. 2000; 74:203-212.
[57] DeCherney A H, diZerega G S. Clinical problem of intraperitoneal postsurgical adhesion formation following general surgery and the use of adhesion prevention barriers. Surg Clin North Am. 1997; 77:671-688.
[58] Ellis H, Moran B J, Thompson J N, Parker M C, Wilson M S, Menzies D, et al. Adhesion-related hospital readmissions after abdominal and pelvic surgery: a retrospective cohort study. Lancet. 1999; 353:1476-1480.
[59] Parker M C, Ellis H, Moran B J, Thompson J N, Wilson M S, Menzies D, et al. Postoperative adhesions: ten-year follow-up of 12,584 patients undergoing lower abdominal surgery. Dis Colon Rectum. 2001; 44:822-829; discussion 829-830.
[60] Holmdahl L, Risberg B. Adhesions: prevention and complications in general surgery. Eur J Surg. 1997; 163:169-174.
[61] Brokelman W, Holmdahl L, Falk P, Klinkenbijl J, Reijnen M. The peritoneal fibrinolytic response to conventional and laparoscopic colonic surgery. J Laparoendosc Adv Surg Tech A. 2009; 19:489-493.
[62] Cheong Y C, Laird S M, Li T C, Shelton J B, Ledger W L, Cooke I D. Peritoneal healing and adhesion formation/reformation. Hum Reprod Update. 2001; 7:556-566.
[63] Ivarsson M L, Bergstrom M, Eriksson E, Risberg B, Holmdahl L. Tissue markers as predictors of postoperative adhesions. Br J Surg. 1998; 85:1549-1554.
[64] Treutner K H, Schumpelick V. [Prevention of adhesions. Wish and reality]. Chirurg. 2000; 71:510-517.
[65] Rout U K, Saed G M, Diamond M P. Expression pattern and regulation of genes differ between fibroblasts of adhesion and normal human peritoneum. Reprod Biol Endocrinol. 2005; 3:1.
[66] Kumar S, Wong P F, Leaper D J. Intra-peritoneal prophylactic agents for preventing adhesions and adhesive intestinal obstruction after non-gynaecological abdominal surgery. Cochrane Database Syst Rev. 2009:CD005080.
[67] Ahmad G, Duffy J M, Farquhar C, Vail A, Vandekerckhove P, Watson A, et al. Barrier agents for adhesion prevention after gynaecological surgery. Cochrane Database Syst Rev. 2008:CD000475.
[68] Binda M M, Hellebrekers B W, Declerck P J, Koninckx P R. Effect of Reteplase and PAI-1 antibodies on postoperative adhesion formation in a laparoscopic mouse model. Surg Endosc. 2009; 23:1018-1025.
[69] Farquhar C, Vandekerckhove P, Watson A, Vail A, Wiseman D. Barrier agents for preventing adhesions after surgery for subfertility. Cochrane Database Syst Rev. 2000:CD000475.
[70] Diamond M P. Reduction of de novo postsurgical adhesions by intraoperative precoating with Sepracoat (HAL-C) solution: a prospective, randomized, blinded, placebo-controlled multicenter study. The Sepracoat Adhesion Study Group. Fertil Steril. 1998; 69:1067-1074.
[71] Gago L A, Saed G M, Chauhan S, Elhammady E F, Diamond M P. Seprafilm (modified hyaluronic acid and carboxymethylcellulose) acts as a physical barrier. Fertil Steril. 2003; 80:612-616.
[72] Mettler L, Audebert A, Lehmann-Willenbrock E, Schive K, Jacobs V R. Prospective clinical trial of SprayGel as a barrier to adhesion formation: an interim analysis. J Am Assoc Gynecol Laparosc. 2003; 10:339-344.
[73] Dunn R, Lyman M D, Edelman P G, Campbell P K. Evaluation of the SprayGel adhesion barrier in the rat cecum abrasion and rabbit uterine horn adhesion models. Fertil Steril. 2001; 75:411-416.
[74] Fazio V W, Cohen Z, Fleshman J W, van Goor H, Bauer J J, Wolff B G, et al. Reduction in adhesive small-bowel obstruction by Seprafilm adhesion barrier after intestinal resection. Dis Colon Rectum. 2006; 49:1-11.
[75] Yagmurlu A, Barlas M, Gursel I, Gokcora I H. Reduction of surgery-induced peritoneal adhesions by continuous release of streptokinase from a drug delivery system. Eur Surg Res. 2003; 35:46-49.
[76] Yeo Y, Bellas E, Highley C B, Langer R, Kohane D S. Peritoneal adhesion prevention with an in situ cross-linkable hyaluronan gel containing tissue-type plasminogen activator in a rabbit repeated-injury model. Biomaterials. 2007; 28:3704-3713.
[77] Hill-West J L, Dunn R C, Hubbell J A. Local release of fibrinolytic agents for adhesion prevention. J Surg Res. 1995; 59:759-763.
[78] Ferland R, Mulani D, Campbell P K. Evaluation of a sprayable polyethylene glycol adhesion barrier in a porcine efficacy model. Hum Reprod. 2001; 16:2718-2723.
[79] diZerega G S, Campeau J D. Peritoneal repair and post-surgical adhesion formation. Hum Reprod Update. 2001; 7:547-555.
[80] Menzies D, Ellis H. The role of plasminogen activator in adhesion prevention. Surg Gynecol Obstet. 1991; 172:362-366.
[81] Harris E S, Morgan R F, Rodeheaver G T. Analysis of the kinetics of peritoneal adhesion formation in the rat and evaluation of potential antiadhesive agents. Surgery. 1995; 117:663-669.
[82] Hellebrekers B W, Trimbos-Kemper T C, Boesten L, Jansen F W, Kolkman W, Trimbos J B, et al. Preoperative predictors of postsurgical adhesion formation and the Prevention of Adhesions with Plasminogen Activator (PAPA-study): results of a clinical pilot study. Fertil Steril. 2009; 91:1204-1214.
[83] Ince A, Eroglu A, Tarhan O, Bulbul M. Peritoneal fibrinolytic activity in peritonitis. Am J Surg. 2002; 183:67-69.
[84] Nordenhem A, Wiman B. Tissue plasminogen activator (tPA) antigen in plasma: correlation with different tPA/inhibitor complexes. Scand J Clin Lab Invest. 1998; 58:475-483.
[85] products Cfvm. Glycerol Formal: Summary Report. In: Agency E M, editor: EMA; 1996.
[86] Spiegel AaN, M M. Use of non-aqueous solvents in parenteral products. J Pharm Sci. 1963; 52:917-927.
[87] Ramchandani M, Robinson D. In vitro and in vivo release of ciprofloxacin from PLGA 50:50 implants. J Control Release. 1998; 54:167-175.
[88] Goepferich A, Shieh L, Langer R. Aspects of polymer erosion. Materials Research Society Symposium Proceedings 1995. p. 155-160.
[89] Lambert W J, Peck K D. Development of an in situ forming biodegradable polylactide-co-glycolide system for controlled release of proteins. J Control Release. 1995; 33:189-195.
[90] Leuneberger H. Physikalische Pharmazie: Wissenschaftliche Verlagsgesellschaft; 2002.

1. Spray system for generating an in situ formed matrix, comprising
a) at least one lipophilic component including at least one polymer based on glycolic acid and lactic acid, and at least one biocompatible solvent with an X log P3 value of less than 0.2, and
b) and at least one hydrophilic component,
wherein the at least two components are present separately from each other prior to application, and are only mixed for or upon spraying, with the components forming a film when sprayed onto human tissue.

2. The spray system of claim 1, further comprising an active agent that is dissolved or dispersed in one of the two components or in both components.

3. The spray system of claim 1, wherein the lipophilic component comprises a PLGA polymer and a biocompatible solvent for the PLGA polymer.

4. The spray system of claim 1, wherein the biocompatible solvent has an X log P3 value of from 0.2 to −1.0.

5. The spray system of claim 1, wherein the biocompatible solvent is selected from tetraglycol, dimethyl isosorbide and/or glycerol formal.

6. The spray system of claim 1, wherein the solvent has an LD₅₀ of at least 1 mg/ml.

7. The spray of claim 1, wherein the polymer is a poly(D,L-lactide-co-glycolide)polymer, wherein the ratio of lactide to glycolide is from 25:75 to 75:25; or the polymer is a PLGA with an intrinsic viscosity value of from 0.16 to 0.70 dl/g; or the PLGA polymer has a molar mass, measured by gel permeation chromatography, of from 10 to 63 kDa.

8. The spray system of claim 1, wherein the polymer is PLGA and the polymer and the biocompatible solvent are selected such that 5 to 60 parts PLGA per 100 parts biocompatible solvent are dissolved.

9. The spray system of claim 1, wherein the hydrophilic component is water, optionally with an active agent dissolved or dispersed therein.

10. The spray system of claim 1, wherein the film formed after spraying has a degradation rate of from 2 to 6 weeks at the site of application.

11. The spray of claim 2, wherein the active agent comprises at least one nucleic acid, comprising RNA, DNA, mRNA, siRNA, miRNA, piRNA, shRNA, antisense-nucleic acid, aptamer, ribozyme, catalytic DNA or a mixture of two or more thereof.

12. The spray system of claim 11, further comprising a nucleic acid encoding a fibrinolytic factor.

13. The spray system of claim 2, wherein the active agent comprises a substance inhibiting the plasminogen activator inhibitor.

14. The spray system of claim 13, wherein the substance inhibiting the plasminogen activator inhibitor is an siRNA.

15. The spray system of claim 11, wherein the nucleic acid is present in a complex with a carrier substance with positively charged groups.

16. The spray system of claim 1, wherein the formed film shows two-phase release kinetics.

17. The spray system comprising polyplexes with an N/P ratio of from 1 to 10.

18. The spray comprising tPA-encoding DNA and PAI inhibitor in a ratio of from 5:1 to 1:5.

19. (canceled)

20. The spray system of claim 12, wherein the fibronolytic factor is tissue plasminogen activator.

21. A method for preventing surgical adhesions or scar formation in a patient, comprising,
applying a film of the spray system of claim 1 to the peritoneum of a patient.


SEQUENCE LISTING (2 sequences)

SEQ ID NO: 1 - RNA, 19 nt, Artificial Sequence
  Description of artificial sequence note = synthetic construct
ggaacaaggaugagaucag

SEQ ID NO: 2 - RNA, 24 nt, Artificial Sequence
  Description of artificial sequence note = synthetic construct
gcaagcugacccugaaguucaumm